(19) United States
(12) Patent Application Publication
(10) Pub. No.: US 2018/0169108 A1
(43) Pub. Date: Jun. 21, 2018

(54) Drug Combinations For the Treatment of HIV

(71) Applicant: The Johns Hopkins University, Baltimore, MD (US)

(72) Inventors: Gregory M. LAIRD, Baltimore, MD (US); Robert SILICIANO, Baltimore, MD (US); Cynthia K. BULLEN, Baltimore, MD (US)

(21) Appl. No.: 15/552,452

(22) PCT Filed: Feb. 19, 2016
(86) PCT No.: PCT/US2016/018577

§ 371 Date: Aug. 21, 2017

Related U.S. Application Data
(60) Provisional application No. 62/139,214, filed on Mar. 27, 2015.
(60) Provisional application No. 62/118,803, filed on Feb. 20, 2015.

(51) Int. Cl. A61K 31/551; A61K 31/22; A61K 31/145; A61K 31/167; A61K 31/4045; A61K 38/15; A61K 31/365; A61K 45/06; A61P 31/18

(52) U.S. Cl. CPC A61K 31/551; A61K 31/22; A61K 31/145; A61K 31/167; A61K 2300/00; A61K 38/15; A61K 31/365; A61K 45/06; A61P 31/18; A61K 31/4045

(57) ABSTRACT

Provided herein are effective latency-reversing agent (LRA) combinations of drugs for the reversal of HIV-1 latency. Such methods utilize, for example, the combination of a protein kinase C agonist and a bromodomain inhibitor combine to reverse latency or a NFκB activator combined with histone deacetylase inhibitors to reverse latency. These findings are surprising considering that many of the individual drug in these combinations show no effect alone. Novel drug combinations for reversal of HIV-1 latency/reactivation of latent HIV-1 may include 1) disulfiram (acetaldehyde dehydrogenase inhibitor, activator of NF-κB via AKT signaling) plus histone deacetylase inhibitors or 2) protein kinase C agonists with bromodomain inhibitor JQ1. Latency-reversing drug combinations for use in HIV-1 infected individuals may eliminate the HIV-1 reservoir for cure/long-term drug-free remission.

### CROSS REFERENCE TO RELATED APPLICATIONS

This application claims priority to U.S. Provisonal Application No: 62/118,803, filed Feb. 20, 2015, and U.S. Provisional Application No. 62/139,214, filed Mar. 27, 2015, the contents of which are incorporated herein by reference in their entirety.

### GOVERNMENT SUPPORT

This invention was made with some government support under grant numbers AI096113, 1U19AI096109, and F31AI116316 awarded by the National Institutes of Health. The government has certain rights in the invention. This statement is included solely to comply with 37 C.F.R. § 401.14(a)(f)(4) and should not be taken as an assertion or admission that the application discloses and/or claims only one invention.

### BACKGROUND

HIV-1 persists in a latent reservoir despite suppressive antiretroviral therapy (ART) (1-5). Resting CD4⁺ T cells (rCD4s) that harbor latent proviruses allow little to no HIV-1 gene expression (6), thereby rendering the virus imperceptible to the host immune response. However, cellular activation reverses this latent state, allowing HIV-1 transcription and subsequent production of replication-competent virus (1-5). This small but stable latent reservoir necessitates life-long ART (7-9) and is a major barrier to curing HIV-1 infection. One proposed strategy for eliminating the latent reservoir is to pharmacologically stimulate HIV-1 gene expression in latently infected cells, rendering these cells susceptible to cytolytic T lymphocytes or viral cytopathic effects (10). While global T cell activation effectively reverses latency, toxicity due to cytokine release precludes its clinical use (11). This has fueled the search for small molecule latency-reversing agents (LRAs) that do not induce T cell activation and cytokine release (reviewed in (12)).

Given the low frequency of latently infected rCD4s in vivo, in vitro models of latency have played a central role in the search for compounds that reactivate latent HIV-1 (compared in (13)). Many LRAs have been identified using these models (13-27). Histone deacetylase (HDAC) inhibitors in particular have shown high latency reversing potential in in vitro models. Pioneering studies by Archin and colleagues have provided some evidence that the HDAC inhibitor vorinostat can perturb HIV-1 latency in vivo (28, 29), and similar results have recently been reported by Rasmussen and colleagues with another HDAC inhibitor, panobinostat (30). However, the magnitude of these effects relative to the total size of the latent reservoir is unclear. When tested in ex vivo assays—which use primary rCD4s recovered directly from HIV-1-infected individuals—these drugs exhibit minimal to modest latency-reversing activity relative to global T cell activation (31-34). These results emphasize that LRAs should be validated by studies using rCD4 from infected individuals. In addition to providing greater physiological relevance than in vitro latency models, primary rCD4s from infected individuals are routinely used in ex vivo viral outgrowth assays that define the size of the latent reservoir in vivo (4, 33, 35).

The inventors recently demonstrated that candidate LRAs including (i) HDAC inhibitors (vorinostat, panobinostat, romidepsin), (ii) disulfiram, which is believed to activate nuclear factor kappa-light-chain-enhancer of activated B cells (NF-κB) and the AKT pathway, and (iii) JQ1, which is a bromo and extra terminal (BET) bromodomain inhibitor, were only minimally active at reversing latency in rCD4s from infected individuals (32). The protein kinase C (PKC) agonist bryostatin-1 was the only single LRA to significantly induce intracellular HIV-1 mRNA production ex vivo (32). This effect, however, was a mere four percent of the maximum reactivation elicited by T cell activation. To assess the activity of LRAs, it is essential to compare their activity relative to both trace baseline levels of HIV-1 gene expression in rCD4s (which vary from individual to individual) and to maximal T cell activation, which serves as a positive control. Maximal T cell activation, used in the viral outgrowth assays with which the latent reservoir was identified, provides an upper bound for latency reversal. LRA regimens that substantially reverse latency ex vivo compared to the benchmark of maximal T cell activation (typically ˜100 fold induction (32)) have not yet been identified. New approaches for latency reversal beyond the use of single LRAs will likely be required for reservoir clearance and a potential cure.

Combinations of mechanistically distinct LRAs may be necessary to overcome the multiple mechanisms governing HIV-1 latency in vivo (36-40). While some combinations have previously been tested in CD4⁺ T cells from infected individuals (37, 41), no comparative ex vivo study has been performed to assess the efficacy of multiple two-drug combinations of leading candidate LRAs. The inventors therefore measured intracellular HIV-1 mRNA levels and supernatant virion production following LRA treatment ex vivo in rCD4s collected from infected individuals on suppressive ART. The inventors identified synergistic drug combinations that reverse latency to levels approaching that of maximal T cell activation. Strikingly, the inventors show here that these robust levels of latency reversal can be achieved without causing functional CD4⁺ T cell activation.

Several clinical trials testing latency reversal by disulfiram or the HDAC inhibitors vorinostat, romidepsin or panobinostat are ongoing or have been completed in patients on art (28-30, 42-44). One indication of successful latency reversal in vivo is a transient increase in plasma HIV-1 RNA, reflecting the release of virus from the latent reservoir. thus far, only romidepsin has been shown to induce detectable increases in plasma HIV-1 RNA using quantitative clinical assays (43). Currently, no quantitative framework exists to predict in vivo responses to LRA treatment using data collected ex vivo. To aid in selecting optimal LRA treatments, a mathematical model was designed to estimate the impact of LRA treatment on in vivo plasma HIV-1 RNA levels based on ex vivo measurements of LRA-induced viral production. With this model, the inventors reconcile the diverse findings of previous in vitro and ex vivo studies and recently reported clinical trial results, highlighting that quantitative analysis of LRA efficacy ex vivo is a useful resource for the design of latency reversing strategies.

### SUMMARY OF THE INVENTION

Provided herein are methods of preventing or treating a HIV infection in a mammal, such as a human. The methods involve administrating to a mammal in need thereof, a therapeutically effective amount of a combination therapy. Such methods comprise administering combinations of latency reversing agents (LRAs) that reverse latency. Such reversal may approach the benchmark of maximal T cell activation of approximately 100-fold induction.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 contains two panels (A) and (B) describing combination LRA treatment robustly increases HIV-1 mRNA expression in rCD4s from infected individuals on ART. Panel (A) depicts intracellular HIV-1 mRNA levels in rCD4s, obtained from infected individuals and treated ex vivo with a single LRA or a combination of two LRAs, presented as fold induction relative to DMSO control. Numbers in parentheses indicate number of individuals used for each treatment. Panel (B) depicts induction of intracellular HIV-1 mRNA by single LRAs, PKC-agonist-containing LRA combinations, and disulfiram-containing LRA combinations presented as a percent of the effect of maximal reactivation with PMA+ ionomycin. Data points represent the mean effect of 2 or 3 replicate LRA treatments of 5 million cells for each individual. For panels A and B, statistical significance was calculated from the HIV-1 mRNA copy number values using a ratio paired T test compared to (A) the DMSO control, (B) bryostatin-1 or prostratin alone, or disulfiram alone. *P<0.05, **P<0.005, ***P<0.0005, ****P<0.00005. Error bars represent SEM.

FIG. 2 depicts PKC agonists synergize with JQ1 and with HDAC inhibitors to significantly increase HIV-1 mRNA expression in rCD4s from infected individuals on ART. Calculation of synergy for LRA combinations using the Bliss independence model. Data are presented as the difference between the observed and predicted fractional response relative to PMA+ionomycin (fraction affected, fₐ,) presented in FIG. 1. See Methods for more detail. Numbers in parentheses indicate number of individuals used for each treatment. Data points represent the mean effect of 2 or 3 replicate LRA treatments of 5 million cells for each individual. Statistical significance for the experimental fₐ was calculated using ratio paired T test compared to the predicted fₐ for each combination. *P<0.05, **P<0.005, ***P<0.0005, ****P<0.00005.

FIG. 3 contains two panels (A) and (B) depicting lower dose of bryostatin-1 synergize with romidepsin to reverse latency. Panel (A) shows intracellular HIV-1 mRNA levels in rCD4s, obtained from infected individuals and treated ex vivo with bryostatin-1 (1 nM or 10 nM) alone or in combination with romidepsin, presented as fold induction relative to DMSO control. Statistical significance was calculated from the HIV-1 mRNA copy number values using a ratio paired T test compared to the DMSO control. *P<0.05, **P<0.005, ***P<0.0005. Panel (B) depicts calculation of synergy for bryostatin-1 (1 nM) and romidepsin using the Bliss independence model. Data are presented as the difference between the observed and predicted fractional response relative to PMA+ionomycin (fraction affected, fₐ,). See Methods for more detail. Statistical significance for the experimental fₐ was calculated using paired T test compared to the predicted fₐ for each combination. *P<0.05. rCD4s from four HIV-1 infected individuals were tested per condition.

FIG. 4 contains three panels (A), (B), and (C) showing PKC agonists alone or in combination with another LRAs induce HIV-1 virus release by rCD4s from infected individuals on ART. HIV-1 virion levels in the culture supernatant of rCD4s from infected individuals 24 hours after addition of a single LRA or a combination of two LRAs, presented as Panel (A) HIV-1 mRNA copies/mL supernatant and Panel (B) as a percent of the effect of maximal reactivation with PMA+ionomycin. Dotted line indicates limit of detection (150 copies per ml). Numbers in parentheses indicate number of individuals used for each treatment. Error bars indicate mean±s.e.m. Statistical significance was calculated from the HIV-1 mRNA copy number values using a ratio paired T test compared to (a) DMSO control, or (b) bryostatin-1 or prostratin alone. Panel (C) shows the calculation of synergy for LRA combinations using the Bliss independence model. Data are presented as the difference between the observed and predicted fraction of supernatant HIV-1 mRNA levels in copies/mL induced by LRA combinations relative to PMA+ionomycin (fraction affected, fₐ). See Methods for more detail. Statistical significance for the experimental fₐ was calculated using a ratio paired T test compared to the predicted fₐ for each combination. *P<0.05, **P<0.005, ***P<0.0005, ****P<0.00005.

FIG. 5 shows the correlation between intracellular and extracellular HIV-1 mRNA after ex vivo LRA treatment. Plot of intracellular HIV-1 mRNA copy number against supernatant HIV-1 mRNA copy number after exposure of rCD4s from the same infected individual to treatments containing (circles) or lacking (triangles) a PKC agonist. For PKC agonist-containing treatments, a statistically significant correlation was demonstrated by Tobit regression analysis (P=0.008 for Chi-squared test) (see Methods).

FIG. 6 contains two panels (A) and (B) showing the effect of LRA treatment on T cell activation-associated surface markers and toxicity. Primary rCD4s treated with a single LRA or a combination of two LRAs were assayed for Panel (A) surface expression of CD25 and CD69 and Panel (B) positivity for annexin V and 7-AAD staining. Data are the mean effect of 2 or 3 independent experiments. Error bars represent SEM.

FIG. 7 contains two panels (A) and (B) showing the PKC agonists alone or in combination with another LRA do not induce substantial cytokine production. Primary Panel (A) rCD4s or Panel (B) PBMCs were treated with a single LRA or a combination of two LRAs were assayed for supernatant cytokine concentrations (pg/mL). Data are the mean effect of 2 or 3 independent experiments.

FIG. 8 contains four panels (A), (B), (C), and (D) depicting mathematical model relating ex vivo virus release to predicted increases in plasma HIV-1 RNA levels in vivo. A viral dynamic model (Panel (A), detailed in Supplementary Methods) was used to estimate changes in plasma HIV-1 RNA levels in response to the LRA treatments for which ex vivo data on virus release was available. Arrows depict routes from latently infected cells, to productively infected cells after exposure to antigen or LRAs. Panel (B) shows the predicted peak plasma HIV-1 RNA levels during LRA treatment. For each LRA treatment, median fold change in supernatant HIV-1 versus the DMSO control (x-axis) was used to estimate LRA-driven activation rate a′; this parameter estimate was used to predict peak plasma viral load following continuous administration of the LRA (y-axis). Panel (C) shows the predicted time-course of viral load (y-axis, log scale) following administration of single-dose LRA treatment that remains active for 1 day. Panel (D) shows the predicted time-course of viral load (y-axis, log scale) following administration of single-dose romidepsin that remains active for 1 day (solid lines) or that continues indefinitely (dotted lines). Gray shading in C, D indicates duration of LRA activity. Parameters: d_{y}=1/day; d′_{y}=1 day⁻¹ (blue curves in B, D, all curves in C) or 1/3 day⁻¹ (red curves in B, D); a+d_{z}=5.2×10⁻⁴ day⁻¹ (reservoir half-life of 44 months), initial viral load=2 copies/mL. Other parameters: See Supplementary Methods. FIG. 9 depicts LRA combinations induce intracellular HIV-1 mRNA production in rCD4s from infected individuals on ART. Intracellular HIV-1 mRNA levels in rCD4s, obtained from infected individuals and treated ex vivo with a single LRA or a combination of two LRAs, presented as copies per million rCD4 equivalents. Numbers in parentheses in FIG. 1A indicate number of individuals used for each treatment.

FIG. 10 contains two panels (A) and (B) depicting that LRA combinations do not increase expression of endogenous housekeeping genes above that caused by a single LRA treatment. Relative expression of Pol2 (A) and G6PD (B) RNA transcripts in rCD4s, obtained from infected individuals (n≥5) and treated ex vivo with a single LRA or a combination of two LRAs, presented as fold induction relative to DMSO control (mean±s.e.m). Numbers in parentheses in FIG. 1A indicate number of individuals used for each treatment.

### DETAILED DESCRIPTION

### A. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “administering” includes any method of delivery of a compound of the present invention, including but not limited to, a pharmaceutical composition, therapeutic agent, or combination thereof, into a subject's system or to a particular region in or on a subject. The phrases “systemic administration,” “administered systemically,” “peripheral administration,” “administered peripherally,” “infusion,” and “reinfusion” as used herein mean the administration of combination therapy, or other material other than directly into the central nervous system, such that it enters the patient's system and, thus, is subject to metabolism and other like processes, for example, subcutaneous administration. “Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The terms “comprise” and “comprising” are used in the inclusive, open sense, meaning that additional elements may be included.

The term “HIV” is known to one skilled in the art to refer to Human Immunodeficiency Virus. There are two types of HIV: HIV-1 and HIV-2. There are many different strains of HIV-1. The strains of HIV-1 can be classified into three groups: the “major” group M, the “outlier” group 0 and the “new” group N. These three groups may represent three separate introductions of simian immunodeficiency virus into humans. Within the M-group there are at least ten subtypes or clades: e.g., clade A, B, C, D, E, F, G, H, I, J, and K. A “clade” is a group of organisms, such as a species, whose members share homologous features derived from a common ancestor. Any reference to HIV-1 in this application includes all of these strains.

The term “including” is used to mean “including but not limited to”. “Including” and “including but not limited to” are used interchangeably.

The term “latency reversing drug combination”, “combination therapy”, or “latency reversing agents” includes but not limited to combinations of the following drugs: Protein Kinase C (PKC) agonists, bromo and external (BET) bromodomain inhibitors, histone deacetylase (HDAC) inhibitors, and acetaldehyde dehydrogenase inhibitor, and activator of nuclear factor kappa-light chain-enhancer of activated B cells (NF-κB) and the AKT pathway. In certain embodiments, the PKC agonist is bryostatin-1, prostratin, ingenol-3-angelate, ingenol mimic, or DAG mimic. In certain embodiments, the acetaldehyde dehydrogenase inhibitor, activator of NF-κB is disulfiram. In certain embodiments, the HDAC inhibitor is selected from the group consisting of vorinostat, panobinostat, and romidepsin. In other embodiments, the HDAC inhibitor is selected from 4-phenylbutyrohydroxamic acid, Acetyldinaline, APHA, Apicidin, AR-42, Belinostat, CUDC-101, CUDC-907, Dacinostat, Depudecin, Droxinostat, Entinostat, Givinostat, HC-Toxin, ITF-2357, JNJ-26481585, KD 5170, LAQ-824, LMK 235, M344, MC1568, MGCD-0103, Mocetinostat, NCH 51, Niltubacin, NSC3852, Oxamflatin, Panobinostat, PCI-24781, PCI-34051, Pracinostat, Pyroxamide, Resminostat, RG2833, RGFP966, Rocilinostat, Romidepsin, SBHA, Scriptaid, Suberohydroxamic acid, Tacedinaline, TC-H 106, TCS HDAC6 20b, Tacedinaline, TMP269, Trichostatin A, Tubacin, Tubastatin A, Valproic acid, or Vorinostat. In certain embodiments, the bromodomain inhibitor is JQ1. In other embodiments, the BET inhibitor is selected from CPI 203, I-BET151, I-BET762, JQ1, MS417, MS436, OTX-015, PFi-1, or RVX-208. In certain embodiments, the latency reversing drug combinations comprise acetaldehyde dehydrogenase inhibitor, activator of NF-κB and the AKT pathway with HDAC inhibitors. In certain embodiments, the latency reversing drug combinations comprise PKC agonists with bromodomain inhibitors. In certain embodiments, the latency reversing drug combinations comprise disulfiram with vorinostate. In certain embodiments, the latency reversing drug combinations comprise disulfiram with panobinostat. In certain embodiments, the latency reversing drug combinations comprise disulfiram with romidepsin. In certain embodiments, the latency reversing drug combinations comprise bryostatin-1 with JQ1. In certain embodiments, the latency reversing drug combinations comprise prostratin with JQ1.

A “patient” or “subject” or “mammal” refers to either a human or non-human animal.

The term “pharmaceutical delivery device” refers to any device that may be used to administer a therapeutic agent or agents to a subject. Non-limiting examples of pharmaceutical delivery devices include hypodermic syringes, multichamber syringes, stents, catheters, transcutaneous patches, microneedles, microabraders, and implantable controlled release devices.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As known to one skilled in the art, “retroviruses” are diploid positive-strand RNA viruses that replicate through an integrated DNA intermediate (proviral DNA). In particular, upon infection by the RNA virus, the lentiviral genome is reverse-transcribed into DNA by a virally encoded reverse transcriptase that is carried as a protein in each retrovirus. The viral DNA is then integrated pseudo-randomly into the host cell genome of the infecting cell, forming a “provirus” which is inherited by daughter cells. The retrovirus genome contains at least three genes: Gag codes for core and structural proteins of the virus; Pol codes for reverse transcriptase, protease and integrase; and Env codes for the virus surface proteins. Within the retrovirus family, HIV is classified as a lentivirus, having genetic and morphologic similarities to animal lentiviruses such as those infecting cats (feline immunodeficiency virus), sheep (visna virus), goats (caprine arthritis-encephalitis virus), and non-human primates (simian immunodeficiency virus).

### B. Methods of Preventing or Treating a HIV Infection

Provided are methods of preventing or treating a lentiviral infection, such as a HIV infection, comprising administering to a mammal in need thereof, a therapeutically effective amount of a latency reversing drug combination.

The term “effective amount” as used herein means an amount effective and at dosages and for periods of time necessary to achieve the desired result. The term “mammal” as used herein includes all members of the animal kingdom including non-humans and humans. In certain embodiments, the mammal may be a human. The human may be afflicted with HIV-1. The human may be chronically infected or acutely infected with HIV-1. The human may be on suppressive antiretroviral therapy.

In certain embodiments, the latency reversing drug combination is administered to the patient more than one time over the course of treating or preventing.

### C. Therapeutically Effective Latency Reversing Combinations

The invention provides methods for treating HIV infection in patients with latency reversing agents combinations.

A. Dose and Dose Regimen

In some embodiments, the total amount of a therapeutically effective substance in a composition to be administered to a patient is one that is suitable for that patient. One of skill in the art would appreciate that different individuals may require different total amounts of the latency reversing combinations. In some embodiments, the amount of the latency reversing agent in the combination is a pharmaceutically effective amount. The skilled worker would be able to determine the amount of the latency reversing agents in a composition needed to treat a patient based on factors such as, for example, the age, weight, and physical condition of the patient. The concentration of the latency reversing agents depends in part on its solubility in the intravenous administration solution and the volume of fluid that can be administered.

In certain embodiments, a latency reversing agent combination, is administered to the subject at a fixed dose ranging from 0.1 mg/m² to 30 mg/m². For example, a latency reversing agent combination may be administered to the subject in a fixed dose of 0.1 mg/m², 0.5 mg/m², 1 mg/m², 3 mg/m², 6 mg/m², 9 mg/m², 12 mg/m², 15 mg/m², 18 mg/m², 21 mg/m², 24 mg/m², 27 mg/m², 30 mg/m², 35 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², etc. Ranges of values between any of the aforementioned recited values are also intended to be included in the scope of the invention, e.g., 0.2 mg/m², 0.6 mg/m², 1.5 mg/m², 2 mg/m², 4 mg/m², 8 mg/m², 10 mg/m², 13 mg/m², 17 mg/m², 20 mg/m², 23 mg/m², 25 mg/m², 26 mg/m², 28 mg/m², 32 mg/m², 45 mg/m², 55 mg/m², 65 mg/m², 75 mg/m², 85 mg/m², 95 mg/m², 105 mg/m², 115 mg/m², 125 mg/m², 135 mg/m², 145 mg/m², 155 mg/m², 165 mg/m², 175 mg/m², 185 mg/m², 195 mg/m², 205 mg/m², as are ranges based on the forementioned doses, e.g., 0.1-5 mg/m², 5-10 mg/m², 10-15 mg/m², 15-20 mg/m², 20-25 mg/m², 25-30 mg/m², 30-80 mg/m², 80-120 mg/m², 120-150 mg/m², 150-175 mg/m², 175-200 mg/m². The total body dose should not exceed 1 g/m² weekly or 200 mg/m² daily.

The concentration of the latency reversing agent combination in the composition administered can be about 1 nM to about 1.5 μM. In certain embodiments, the concentration of the latency reversing agent, alone or in combination, may be about 1 nM, about 5 nM, about 10 nM, about 20 nM, about 40 nM, about 50 nM, about 60 nM, about 75 nM, about 100 nM, about 125 nM, about 150 nM, about 175 nM, about 200 nM, about 225 nM, about 250 nM, about 275 nM, about 300 nM, about 325 nM, about 350 nM, about 375 nM, about 400 nM, about 425 nM, about 450 nM, about 475 nM, about 500 nM, about 525 nM, about 550 nM, about 575 nM, about 600 nM, about 625 nM, about 650 nM, about 675 nM, about 700 nM, about 725 nM, about 750 nM, about 775 nM, about 800 nM, about 825 nM, about 850 nM, about 875 nM, about 900 nM, about 925 nM, about 950 nM, about 1 μM, about 1.1 μM, about 1.2 μM, about 1.3 μM, about 1.4 μM, about 1.5 μM, etc. In certain embodiments, the concentration of bryostatin-1 may be about 10 nM. In certain embodiments, the concentration of prostratin may be about 300 nM. In certain embodiments, the concentration of disulfiram may be about 500 nM. In certain embodiments, the concentration of JQ1 may be about 1 μM. In certain embodiments, the concentration of panobinostat may be about 30 nM. In certain embodiments, the concentration of romidepsin may be about 40 nM. In certain embodiments, the concentration of vorinostat may be about 335 nM. In certain embodiments, the concentration of prostratin may be about 300 nM. The latency reversing agent combination can be administered at a rate sufficient to achieve an increase or modulation in one or more physiological parameters, such as HIV-1 mRNA levels, latency reversal in rCD4s from infected individual, intracellular HIV-1 RNA levels, intracellular HIV-1 protein levels, extracellular HIV-1 RNA levels, extracellular HIV-1 protein levels, or plasma viral load. The latency reversing agent combination can be administered at a rate sufficient to achieve a decrease or modulation in one or more physiological parameters relating to HIV-1 latency, such as HIV-1 proviral DNA levels, the levels of intact HIV-1 proviruses, the levels of replication competent HIV-1 proviruses, the frequency of latently infected cells. The latency reversing agent combination can be administered at a rate sufficient to achieve a significant delay in viral rebound after cessation of antiretroviral therapy, indicative of a decrease in the size of the HIV-1 latent reservoir. A patient may be coupled to a monitor that provides continuous, periodic, or occasional measurements during some or all of the course of treatment. The rate of administration may be modulated manually (e.g., by a physician or nurse) or automatically (e.g., by a medical device capable of modulating delivery of the composition in response to physiological parameters received from the monitor) to maintain the patient's physiological and/or biomarker parameters within a desired range or above or below a desired threshold, for example, the rate of administration of the latency reversing agent combination may be from about 0.032 ng/kg/min to about 100 ug/kg/min in the injectable composition. In some embodiments, the rate of administration of the latency reversing agent combination may be from about 0.4 to about 45 ug/min, from about 0.12 to about 19 ug/min, from about 3.8 to about 33.8 ug/min, from about 0.16 to about 2.6 ug/min, etc. In particular embodiments, the rate of administration of the latency reversing agent combination may be about 0.032 ng/kg/min, about 0.1 ng/kg/min, about 0.32 ng/kg/min, about 1 ng/kg/min, about 1.6 ng/kg/min, about 2 ng/kg/min, about 3 ng/kg/min, about 4 ng/kg/min, about 5 ng/kg/min, about 6 ng/kg/min, about 7 ng/kg/min, about 8 ng/kg/min, about 9 ng/kg/min, about 10 ng/kg/min, about 15 ng/kg/min, about 20 ng/kg/min, about 25 ng/kg/min, about 30 ng/kg/min, about 40 ng/kg/min, about 50 ng/kg/min, about 60 ng/kg/min, about 70 ng/kg/min, about 80 ng/kg/min, about 90 ng/kg/min, about 100 ng/kg/min, about 200 ng/kg/min, about 300 ng/kg/min, about 400 ng/kg/min, about 500 ng/kg/min, about 600 ng/kg/min, about 700 ng/kg/min, about 800 ng/kg/min, about 900 ng/kg/min, about 1 ug/kg/min, about 1.1 ug/kg/min, about 1.2 ug/kg/min, about 1.3 ug/kg/min, about 1.4 ug/kg/min, about 1.5 ug/kg/min, about 1.5 ug/kg/min, about 1.6 ug/kg/min, about 1.7 ug/kg/min, about 1.8 ug/kg/min, about 1.9 ug/kg/min, about 2 ug/kg/min, about 2.1 ug/kg/min, about 2.2 ug/kg/min, about 2.3 ug/kg/min, about 2.4 ug/kg/min, about 2.5 ug/kg/min, about 2.6 ug/kg/min, about 2.7 ug/kg/min, about 2.8 ug/kg/min, about 2.9 ug/kg/min, about 3.0 ug/kg/min, about 3.1 ug/kg/min, about 3.2 ug/kg/min, about 3.3 ug/kg/min, about 3.4 ug/kg/min, about 3.5 ug/kg/min, about 3.6 ug/kg/min, about 3.7 ug/kg/min, about 3.8 ug/kg/min, about 3.9 ug/kg/min, about 4.0 ug/kg/min, about 4.1 ug/kg/min, about 4.2 ug/kg/min, about 4.3 ug/kg/min, about 4.4 ug/kg/min, about 4.5 ug/kg/min, about 4.6 ug/kg/min, about 4.7 ug/kg/min, about 4.8 ug/kg/min, about 4.9 ug/kg/min, about 5.0 ug/kg/min, about 6 ug/kg/min, about 7 ug/kg/min, about 8 ug/kg/min, about 9 ug/kg/min, about 10 ug/kg/min, about 11 ug/kg/min, about 12 ug/kg/min, about 13 ug/kg/min, about 14 ug/kg/min, about 15 ug/kg/min, about 16 ug/kg/min, about 17 ug/kg/min, about 18 ug/kg/min, about 19 ug/kg/min, about 20 ug/kg/min, about 25 ug/kg/min, about 30 ug/kg/min, about 31 ug/kg/min, about 32 ug/kg/min, about 33 ug/kg/min, about 33.8 ug/kg/min, about 34 ug/kg/min, about 35 ug/kg/min, about 40 ug/kg/min, about 45 ug/kg/min, about 50 ug/kg/min, about 55 ug/kg/min, about 60 ug/kg/min, about 65 ug/kg/min, about 70 ug/kg/min, about 75 ug/kg/min, about 80 ug/kg/min, about 85 ug/kg/min, about 90 ug/kg/min, about 95 ug/kg/min, about 100 ug/kg/min, etc.

The composition may be administered over a period of time selected from at least 8 hours; at least 24 hours; and from 8 hours to 24 hours. The composition may be administered continuously for at least 2-6 days, such as 2-11 days, continuously for 2-6 days, for 8 hours a day over a period of at least 2-6 days, such as 2-11 days. A weaning period (from several hours to several days) may be beneficial after prolonged infusion. In certain embodiments, the duration of treatment may last up to 8 consecutive weeks of dosing or until the development of dose-limiting toxicity.

B. Pharmaceutical Formulations

The compositions of the invention can be administered through any suitable route. In some embodiments, the compositions of the invention are suitable for parenteral administration. These compositions may be administered, for example, intraperitoneally, intravenously, intrarenally, or intrathecally. In some embodiments, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

The compositions may be solutions containing at least 0.5%, 1%, 5% or 10% by weight of the latency reversing agent combination, e.g., up to about 10% or 15% by weight. In certain embodiments, the latency reversing agent combination is provided as a colloidal solution in water. The size of the colloidal particles may be less than 1 μm in diameter, preferably less than about 0.65 μm, and most preferably less than about 0.2 μm.

The formulation may comprise suitable excipients including pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for intravaginal or intrarectal administration, pessaries, suppositories, creams or foams. Preferably, the route of administration is parenteral, more preferably intravenous.

In alternative embodiments, a pharmaceutical composition of the invention may be in a form adapted for oral dosage, such as for example a syrup or palatable solution; a form adapted for topical application, such as for example a cream or ointment; or a form adapted for administration by inhalation, such as for example a microcrystalline powder or a solution suitable for nebulization. Methods and means for formulating pharmaceutical ingredients for alternative routes of administration are well-known in the art, and it is to be expected that those skilled in the relevant arts can adapt these known methods to the latency reversing agent combination of the invention.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention may optionally be scored or prepared with coatings and shells, such as enteric coatings and other coatings well known in the pharmaceutical-formulating art. They may also be formulated so as to provide slow or controlled release of the modified therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions that can be used include polymeric substances and waxes. The latency reversing agent combination can also be in micro-encapsulated form, if appropriate, with one or more of the above-described excipients.

Liquid dosage forms for oral administration of the latency reversing agent combination of the invention include pharmaceutically acceptable emulsions, microemulsions, solutions, suspensions, syrups and elixirs. In addition to the latency reversing agent combination, the liquid dosage forms may contain inert diluents commonly used in the art, such as, for example, water or other solvents, solubilizing agents and emulsifiers.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Administration of medicament may be indicated for the treatment of mild, moderate or severe acute or chronic symptoms or for prophylactic treatment. It may be appreciated that the precise dose administered may depend on the age and condition of the patient, the particular particulate medicament used and the frequency of administration and may ultimately be at the discretion of the attendant physician. Typically, administration may occur weekly, though may occur at a regular or irregular frequency, such as daily or monthly or a combination thereof (e.g., daily for five days once a month).

Pharmaceutical compositions of this invention suitable for parenteral administration comprise a latency reversing agent combination of the invention in combination with one or more pharmaceutically acceptable sterile isotonic aqueous or non-aqueous solutions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Examples of pharmaceutically acceptable antioxidants include but are not limited to ascorbic acid, cysteine hydrochloride, sodium metabisulfite, sodium sulfite, ascorbyl palmitate, butylated hydroxyanisole (BHA), butylated hydroxytoluene (BHT), propyl gallate, alpha-tocopherol, and chelating agents such as citric acid, ethylenediamine tetraacetic acid (EDTA), sorbitol, tartaric acid, phosphoric acid, and the like.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Dosage forms for the topical or transdermal administration of a compound of this invention include powders, sprays, ointments, pastes, creams, lotions, gels, solutions, patches and inhalants. The latency reversing agent combination may be mixed under sterile conditions with a pharmaceutically acceptable carrier, and with any preservatives, buffers, or propellants that may be required.

A pH-adjusting agent may be beneficial to adjust the pH of the compositions by including a pH-adjusting agent in the compositions of the invention. Modifying the pH of a formulation or composition may have beneficial effects on, for example, the stability or solubility of a therapeutically effective substance, or may be useful in making a formulation or composition suitable for parenteral administration. pH-adjusting agents are well known in the art. Accordingly, the pH-adjusting agents described herein are not intended to constitute an exhaustive list, but are provided merely as exemplary pH-adjusting agents that may be used in the compositions of the invention. pH-adjusting agents may include, for example, acids and bases. In some embodiments, a pH-adjusting agent includes, but is not limited to, acetic acid, hydrochloric acid, phosphoric acid, sodium hydroxide, sodium carbonate, and combinations thereof. The pH of the compositions of the invention may be any pH that provides desirable properties for the formulation or composition. Desirable properties may include, for example, therapeutically effective substance stability, increased therapeutically effective substance retention as compared to compositions at other pHs, and improved filtration efficiency. In some embodiments, the pH of the compositions of the invention may be from about 3.0 to about 9.0, e.g., from about 5.0 to about 7.0. In particular embodiments, the pH of the compositions of the invention may be 5.5±0.1, 5.6±0.1, 5.7±0.1, 5.8±0.1, 5.9±0.1, 6.0±0.1, 6.1±0.1, 6.2±0.1, 6.3±0.1, 6.4±0.1, or 6.5±0.1.

In certain embodiments, the latency reversing agent combination is prepared substantially ethanol-free and suitable for parenteral administration. By substantially free of ethanol, it is meant that the compositions of the invention contain less than 5% ethanol by weight. In preferred embodiments the compositions contain less than 2%, and more preferably less than 0.5% ethanol by weight. In certain embodiments, the compositions further comprise one or more pharmaceutically acceptable excipients. Such compositions include aqueous solutions of the latency reversing agent combination of the invention. In certain embodiments of such aqueous solutions, the latency reversing agent combination occurs at a concentration of at least 7 mg/mL, at least 10, or 15 or more mg/ml. Any of such compositions are also substantially free of organic solvents other than ethanol.

A buffer may be used to resuspend the compound in solution. In certain embodiments, a buffer may have a pKa of, for example, about 5.5, about 6.0, or about 6.5. One of skill in the art would appreciate that an appropriate buffer may be chosen for inclusion in compositions of the invention based on its pKa and other properties. Buffers are well known in the art. Accordingly, the buffers described herein are not intended to constitute an exhaustive list, but are provided merely as exemplary buffers that may be used in the compositions of the invention. In certain embodiments, a buffer may include one or more of the following: Tris, Tris HCl, potassium phosphate, sodium phosphate, sodium citrate, sodium ascorbate, combinations of sodium and potassium phosphate, Tris/Tris HCl, sodium bicarbonate, arginine phosphate, arginine hydrochloride, histidine hydrochloride, cacodylate, succinate, 2-(N-morpholino)ethanesulfonic acid (MES), maleate, bis-tris, phosphate, carbonate, and any pharmaceutically acceptable salts and/or combinations thereof.

A solubilizing agent may be added to increase the solubility of a drug or compound. In some embodiments, it may be beneficial to include a solubilizing agent to the latency reversing agent combination. Solubilizing agents may be useful for increasing the solubility of any of the components of the formulation or composition, including a therapeutically effective latency reversing agent combination or an excipient. The solubilizing agents described herein are not intended to constitute an exhaustive list, but are provided merely as exemplary solubilizing agents that may be used in the compositions of the invention. In certain embodiments, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

A stabilizing agent may help to increase the stability of a therapeutically effective substance in compositions of the invention. This may occur by, for example, reducing degradation or preventing aggregation of a therapeutically effective substance. Without wishing to be bound by theory, mechanisms for enhancing stability may include sequestration of the therapeutically effective substance from a solvent or inhibiting free radical oxidation of the anthracycline compound. Stabilizing agents are well known in the art. Accordingly, the stabilizing agents described herein are not intended to constitute an exhaustive list, but are provided merely as exemplary stabilizing agents that may be used in the compositions of the invention. Stabilizing agents may include, but are not limited to, emulsifiers and surfactants.

A surfactant may be added to reduce the surface tension of a liquid composition. This may provide beneficial properties such as improved ease of filtration. Surfactants also may act as emulsifying agents and/or solubilizing agents. Surfactants are well known in the art. Accordingly, the surfactants described herein are not intended to constitute an exhaustive list, but are provided merely as exemplary surfactants that may be used in the compositions of the invention. Surfactants that may be included include, but are not limited to, sorbitan esters such as polysorbates (e.g., polysorbate 20 and polysorbate 80), lipopolysaccharides, polyethylene glycols (e.g., PEG 400 and PEG 3000), poloxamers (i.e., pluronics), ethylene oxides and polyethylene oxides (e.g., Triton X-100), saponins, phospholipids (e.g., lecithin), and combinations thereof.

A tonicity-adjusting reagent may be used to help make a formulation or composition suitable for administration. The tonicity of a liquid composition is an important consideration when administering the composition to a patient, for example, by parenteral administration. Tonicity-adjusting agents are well known in the art. Accordingly, the tonicity-adjusting agents described herein are not intended to constitute an exhaustive list, but are provided merely as exemplary tonicity-adjusting agents that may be used in the compositions of the invention. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

In certain embodiments, a therapeutically effective amount of a latency reversing drug combination comprises NFκB activator and HDAC inhibitor. In other embodiments, a therapeutically effective amount of a latency reversing drug combination comprises PKC agonists and bromodomain inhibitor. In certain embodiments, a therapeutically effective amount of a latency reversing drug combination comprises disulfiram with vorinostat, disulfiram with panobinostat, or disulfiram with romidepsin. In certain embodiments, a therapeutically effective amount of a latency reversing drug combination comprises bryostatin-1 with JQ1 or prostratin with JQ1.

The amount of active ingredient that may be combined with the carrier materials to produce a single dosage form will vary depending upon the host treated and the particular mode of administration. The dose of the combination therapy may vary according to factors such as the infection state, age, sex, and weight of the individual, and the latency reversing agent to elicit a desired response in the individual. Dosage regime may be adjusted to provide the optimum therapeutic response. For example, several divided doses may be administered daily or the dose may be proportionally reduced as indicated by the exigencies of the therapeutic situation. The dose of the latency reversing agent may also be varied to provide optimum preventative or treatment dose response depending upon the circumstances.

In certain embodiments, the efficacy of the prevention or treatment of the methods of the present invention may be determined from samples obtained from the mammal after treatment has began using the following. In certain embodiments, the efficacy is determined comparing a sample of a mammal obtained during the course of treatment to a sample which has been previously obtained from the patient, such as at the start of treatment or in an initial sample obtained two, three, or four weeks post HIV infection but prior to treatment. In certain embodiments, the levels of levels of intracellular HIV-1 mRNA when compared to a sample previously obtained from the mammal prior to initiation of treatment. In certain embodiments, the increase in the levels of intracellular HIV-1 mRNA is measured as about 2-, about 4-, about 6-, about 8-, about 10-, about 12-, or about 14-fold increase compared to control. In other embodiments, the efficacy of the treatment results in an increase in the production and release of HIV-1 virions compared to control. In certain embodiments, the increase in the production and release of HIV-1 virions is measured as an average of about 1, about 1.5, about 2, about 2.5, about 3, about 3.5, or about 4×10⁵ HIV-1 mRNA copies per milliliter of supernatant. In certain embodiments, the efficacy of the treatment results in an increase in T cell activation. In certain embodiments, the increase in T cell activation is measured as an increase in the production and release of at least one proinflammatory cytokine compared to control. In certain embodiments, the proinflammatory cytokine is selected from the group consisting of TNF-α, IFN-γ, IL-2, IL-4, IL-6, IL-10, and IL-17. In other embodiments, the efficacy of the treatment results in an increase in the levels of plasma HIV-1 RNA. In certain embodiments, the increase of the levels of plasma HIV-1 RNA is in measure as about 2-logs, 3-logs, 4-logs, 5-logs, 6-logs, 7-logs, 8-logs, or 9-logs increase compare to control.

All references cited herein are all incorporated by reference herein, in their entirety, whether specifically incorporated or not. All publications, patents, or patent applications cited herein are hereby expressly incorporated by reference for all purposes. In case of conflict, the definitions within the instant application govern.

Having now fully described this invention, it will be appreciated by those skilled in the art that the same can be performed within a wide range of equivalent parameters, concentrations, and conditions without departing from the spirit and scope of the invention and without undue experimentation.

The present description is further illustrated by the following examples, which should not be construed as limiting in any way.

### Examples

Reversal of HIV-1 latency by small molecules is a potential cure strategy. This approach will likely require effective drug combinations to achieve high levels of latency reversal. Using resting CD4⁺ T cells (rCD4s) from infected individuals, the inventors herein developed an experimental and theoretical framework to identify effective latency-reversing agent (LRA) combinations. Utilizing ex vivo assays for intracellular HIV-1 mRNA and virion production, the inventors compared two-drug combinations of leading candidate LRAs and identified multiple combinations that effectively reverse latency. Notably, the inventors showed that protein kinase C agonists in combination with JQ1 or histone deacetylase inhibitors robustly induce HIV-1 transcription and virus production when directly compared to maximum reactivation by T cell activation. Using the Bliss independence model to quantitate combined drug effects, it was demonstrated that these combinations synergize to induce HIV-1 transcription. This robust latency reversal occurs without release of proinflammatory cytokines by rCD4s. To extend the clinical utility of the findings provided herein, the inventors applied a mathematical model that estimates in vivo changes in plasma HIV-1 RNA from ex vivo measurements of virus production. This study reconciles diverse findings from previous studies, establishes a quantitative experimental approach to evaluate combinatorial LRA efficacy, and presents a model to predict in vivo responses to LRAs.

### Results:

Quantifying the combined effects of two or more LRAs requires first understanding the effect of each drug alone. Therefore, five million purified rCD4s from infected individuals on suppressive ART (participant characteristics in Table 1) were treated with single LRAs or vehicle alone for 24 hours and then measured levels of intracellular HIV-1 mRNA using a primer/probe set that detects the 3′ sequence common to all correctly terminated HIV-1 mRNAs (32, 45).

TABLE 1
Characteristics of HIV-1 infected study participants.

| | | | | | | | Time on | |
|---|---|---|---|---|---|---|---|---|
| | | | | Duration of | | Time on | suppressive | Peak reported |
| | | | | infection | | ART | ART | viral load |
| Pt. ID | Age | Sex | Race | (months) | ART regimen | (months) | (months) | (copies mL⁻¹) |
| S1 | 45 | M | W | 89 | EFV/FTC/TDF | 87 | 40 | 60070 |
| S2 | 42 | M | B | 236 | EFV/FTC/TDF | 129 | 65 | 507612 |
| S3 | 48 | M | B | 293 | RAL/DRV/r | 149 | 28 | 66 |
| S4 | 52 | M | B | 221 | ABC/3TC/ATV/r | 197 | 8 | 67555 |
| S5 | 54 | M | B | 149 | RAL/FTC/DRV/v | 96 | 9 | >750000 |
| S6 | 47 | F | B | 197 | FTC/TDF/ATV/r | 118 | 106 | 36276 |
| S7 | 49 | M | W | 136 | ABC/3TC/RAL | 135 | 134 | 10414 |
| S8 | 60 | M | B | 89 | RAL/3TC/DRV/r | 56 | 49 | 151114 |
| S9 | 52 | M | B | 137 | RAL/3TC/DRV/r | 135 | 41 | 739349 |
| S10 | 56 | M | B | 136 | FTC/TDF/EVG/c | 135 | 32 | 10485 |
| S11 | 55 | F | B | 136 | FTC/TDF/EVG/c | 136 | 108 | 158523 |
| S12 | 31 | M | W | 100 | FTC/TDF/EFV | 78 | 65 | 74934 |
| S13 | 52 | M | B | 216 | EFV/DRV/r/RAL | 192 | 16 | n/a |
| S14 | 62 | F | B | 193 | ABC/3TC/ATV/r | 129 | 21 | 53,327 |

Abbreviations:
male (M),
female (F),
Caucasian/white (W),
African American/Black (B),
abacavir (ABC),
emtricitabine (FTC),
lamivudine (3TC),
tenofovir (TDF),
efavirenz (EFV),
etravirine (ETR),
nevirapine (NVP),
atazanavir boosted with ritonavir (ATV/r),
darunavir boosted with ritonavir (DRV/r),
fosamprenavir boosted with ritonavir (FPV/r),
lopinavir boosted with ritonavir (LPV/r),
elvitegravir boosted with cobicistat (EVG/c),
raltegravir (RAL),
maraviroc (MVC)

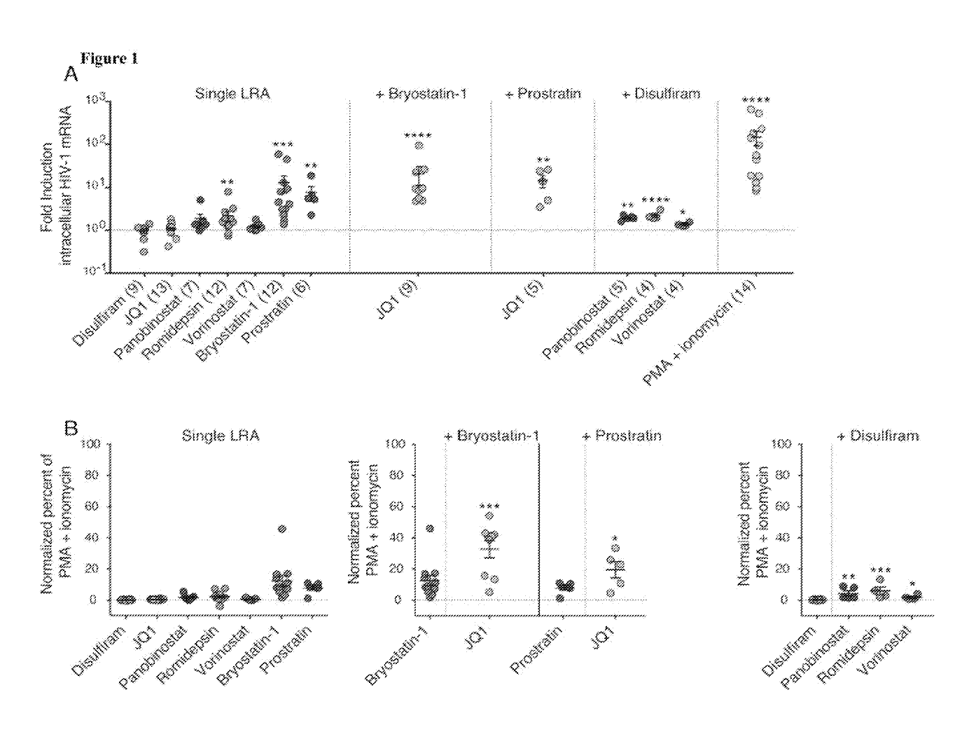

Drugs were used at concentrations previously shown to be effective at reversing latency in model systems. Of the LRAs tested individually, only the HDAC inhibitor romidepsin and the PKC agonists bryostatin-1 and prostratin caused statistically significant increases in intracellular HIV-1 mRNA (mean increases of 2.2, 12.8, and 7.7-fold respectively, FIG. 1A, FIG. 9). In contrast, the T cell activation control of PMA+ionomycin (PMA/I) dramatically elevated levels of intracellular HIV-1 mRNA (mean increase of 148.8-fold, FIG. 1A). Treatment of CD4 T cells with PMA/I causes a dramatic up-regulation of numerous signaling pathways downstream of the T cell receptor, many of which promote efficient HIV-1 transcription. When LRA-induced increases in HIV-1 mRNA are normalized as a percent of the effect elicited by T cell activation with PMA/I, it is apparent that individual LRAs generally show limited efficacy ex vivo (FIG. 1B).

To identify effective two-drug combinations of LRAs, rCD4s from infected individuals on suppressive ART were treated with bryostatin-1, prostratin, or disulfiram in combination with a mechanistically distinct LRA. Ten of the 11 combinations tested caused a significant increase in intracellular HIV-1 mRNA relative to the DMSO control (FIG. 1A, FIG. 9). To compare the efficacy of these combinations, increases in intracellular HIV-1 mRNA levels were plotted as a percent of the effect of the T cell activation control, PMA/I. Combinations of the PKC agonist bryostatin-1 with JQ1 or with each of three different HDAC inhibitors were significantly more effective than bryostatin-1 alone (FIG. 1B), with some combinations approaching the magnitude of induction stimulated by T cell activation with PMA/I. For example, treatment with a combination of bryostatin-1 and panobinostat caused increases in intracellular HIV-1 mRNA that were on average 51.5% of that seen with the PMA/I control, with increases of 89.1% seen in some infected individuals. Similarly, treatment with a combination of bryostatin-1 and JQ1 caused increases in intracellular HIV-1 mRNA that were on average 32.6% of that seen with the PMA/I control. Combinations of the PKC agonist prostratin with JQ1 or romidepsin produced increases in HIV-1 RNA that were significantly greater than those seen with prostratin alone. Two-drug combinations containing disulfiram and an HDAC inhibitor were significantly more active that either compound alone. However, the observed induction of intracellular HIV-1 mRNA did not exceed 14% of the PMA/I response (FIG. 1B).

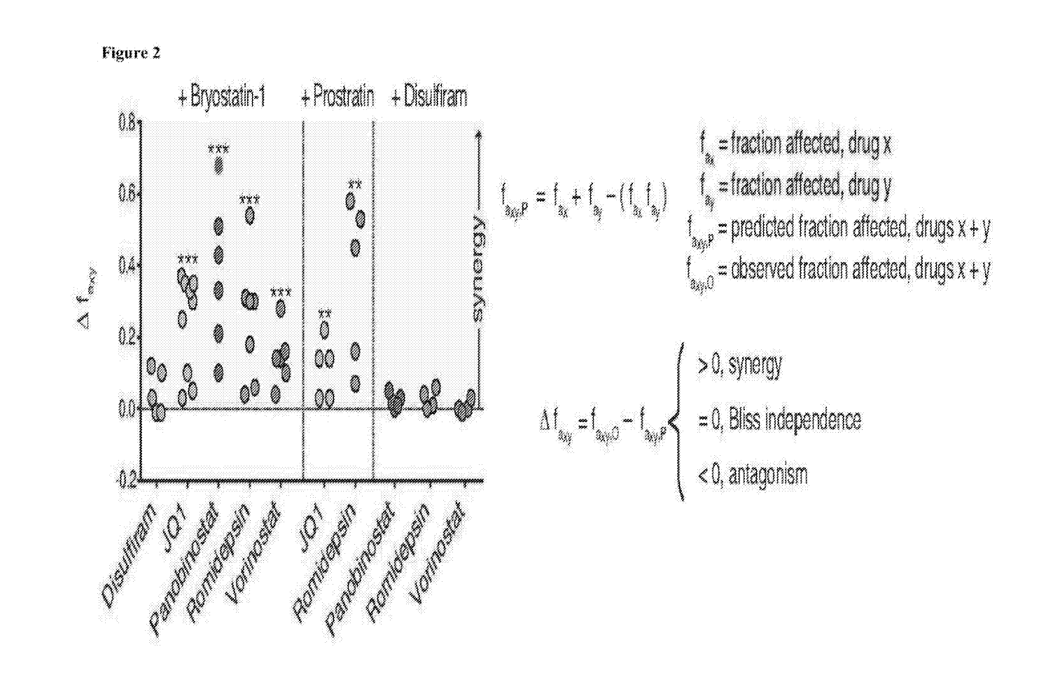

Commonly used models for determining whether drugs act synergistically are based on the assumption that the drugs act through the same mechanism, an assumption that does not apply to combinations of LRAs (46). To quantitate interactions between LRAs, the experimentally observed combined effects was compared to the effects predicted under the Bliss independence model for combined drug effects (47) (FIG. 2). This model assumes that compounds act through different mechanisms, such that their effects multiply when administered in combination. A drug combination whose effect significantly exceeds that predicted by the Bliss model can be said to exhibit synergy. It was found that the PKC agonists synergize significantly with JQ1 and the HDAC inhibitors to induce intracellular HIV-1 mRNA ex vivo (FIG. 2). Disulfiram containing combinations did not exhibit synergy, but rather conformed to the predictions of the Bliss independence model (FIG. 2).

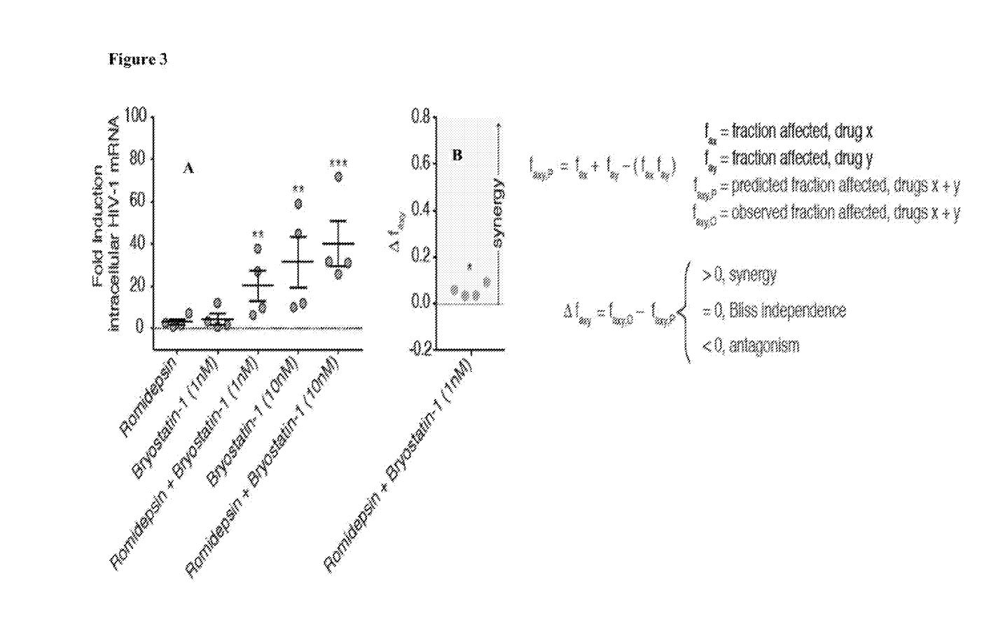

To further explore the synergistic relationship between bryostatin-1 and the HDAC inhibitors, a ten-fold lower concentration of bryostatin-1 alone and in combination with the HDAC inhibitor romidepsin was tested. Treatment with 1 nM bryostatin-1 did not induce significant intracellular HIV-1 mRNA. However, when 1 nM bryostatin-1 was combined with romidepsin, significant induction of intracellular HIV-1 mRNA was observed (FIG. 3A, mean 20.2 fold induction) and this combination was synergistic (FIG. 3B).

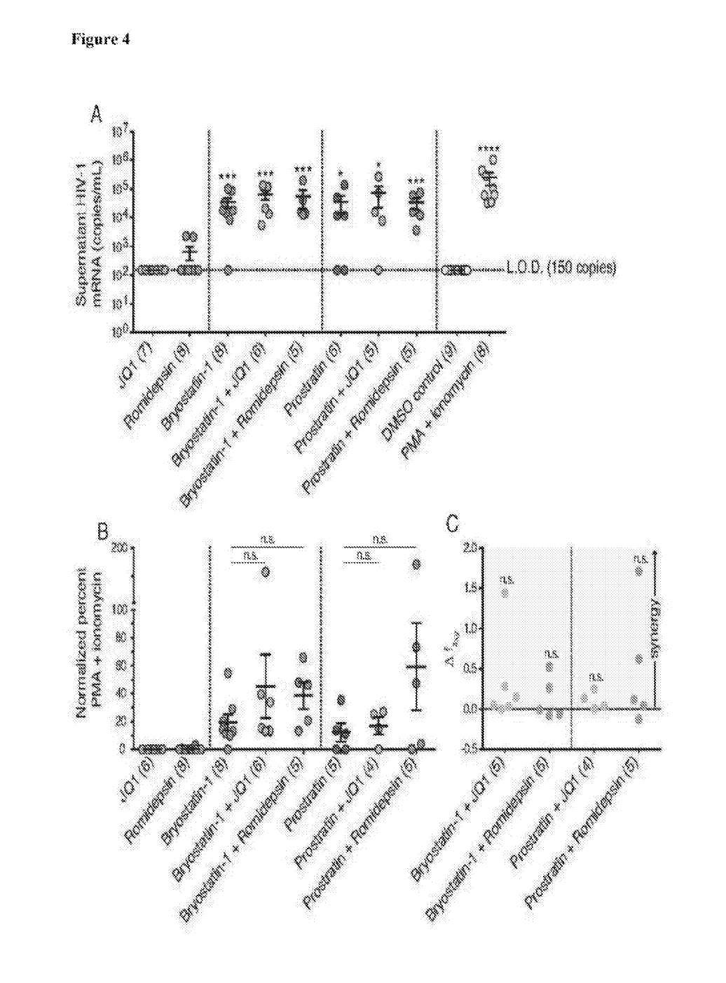

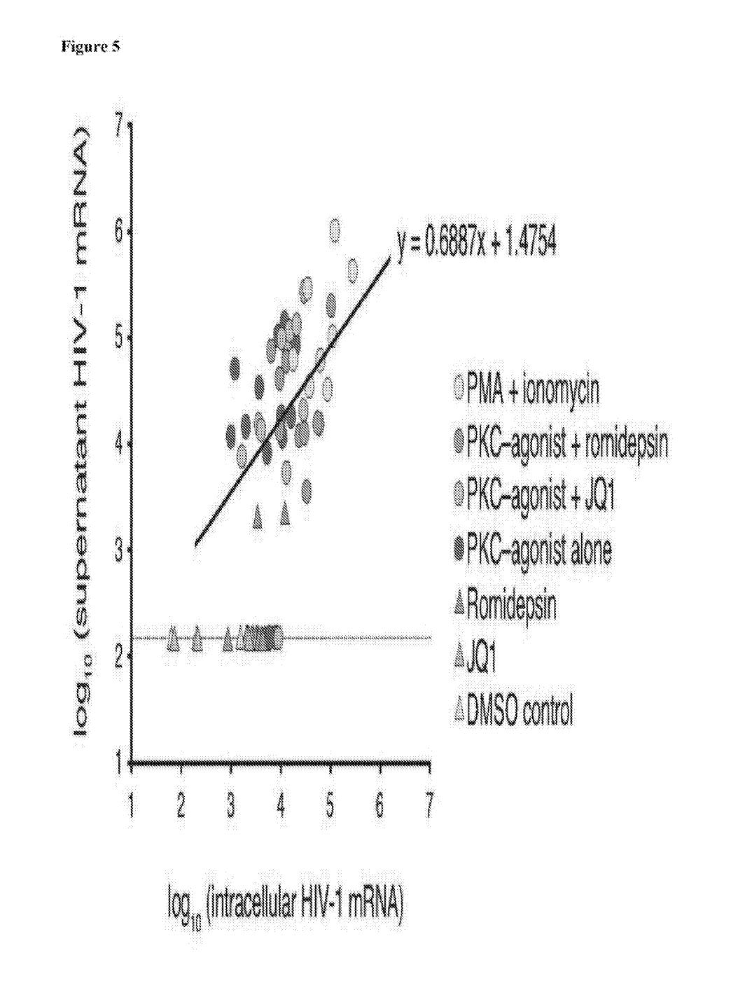

Production and release of HIV-1 virions by LRA-treated rCD4s indicates complete reversal of latency in those cells. To assess whether combinations of LRAs induced rapid virus release, HIV-1 mRNA were measured in the culture supernatants of LRA-treated rCD4s from infected individuals on suppressive ART using an RT-qPCR assay previously shown to provide sensitive and accurate quantitation of HIV-1 virion production (45). LRAs showing synergistic effects were focused on, particularly JQ1, romidepsin, and the PKC agonists bryostatin-1 and prostratin. No virus production was observed after 24 hours of treatment with the DMSO control in any of the individuals tested (limit of detection=150 copies HIV-1 RNA/mL supernatant) whereas treatment with PMA/I induced an average of 2.6×10⁵ HIV-1 mRNA copies/mL supernatant. Of the LRAs tested, only bryostatin-1 and prostratin induced significant virus release as single agents (FIG. 4A,B). Combinations of bryostatin-1 or prostratin with JQ1 or romidepsin also caused significant virus release (FIG. 4A,B), but the combined effects did not significantly exceed those of bryostatin-1 or prostratin alone (FIG. 4B). Surprisingly, combination LRA treatment exceeded the effect seen with maximal T cell activation by PMA/I in some instances (FIG. 4B). The Bliss independence model was applied to quantitate interactions between LRAs. While synergy was observed in some individuals, collectively the combined LRA effects on virus production did not significantly exceed those predicted by the Bliss independence model (FIG. 4C).

Next, the relationship between intracellular HIV-1 mRNA levels and HIV-1 virion production by LRA-treated rCD4s was examined (FIG. 5). Treatments including the PKC agonists bryostatin-1 or prostratin clustered with PMA (also a PKC agonist)+ionomycin while treatments lacking a PKC agonist showed much lower activity, especially with regards to virion production. Tobit regression analysis of only the treatments containing a PKC agonist yielded a significant correlation between increases in intracellular HIV-1 mRNA and virion release (FIG. 5, P=0.008 for Chi-squared test). Thus with respect to inducing virion production from latently infected cells, PKC agonists appear to be of particular importance.

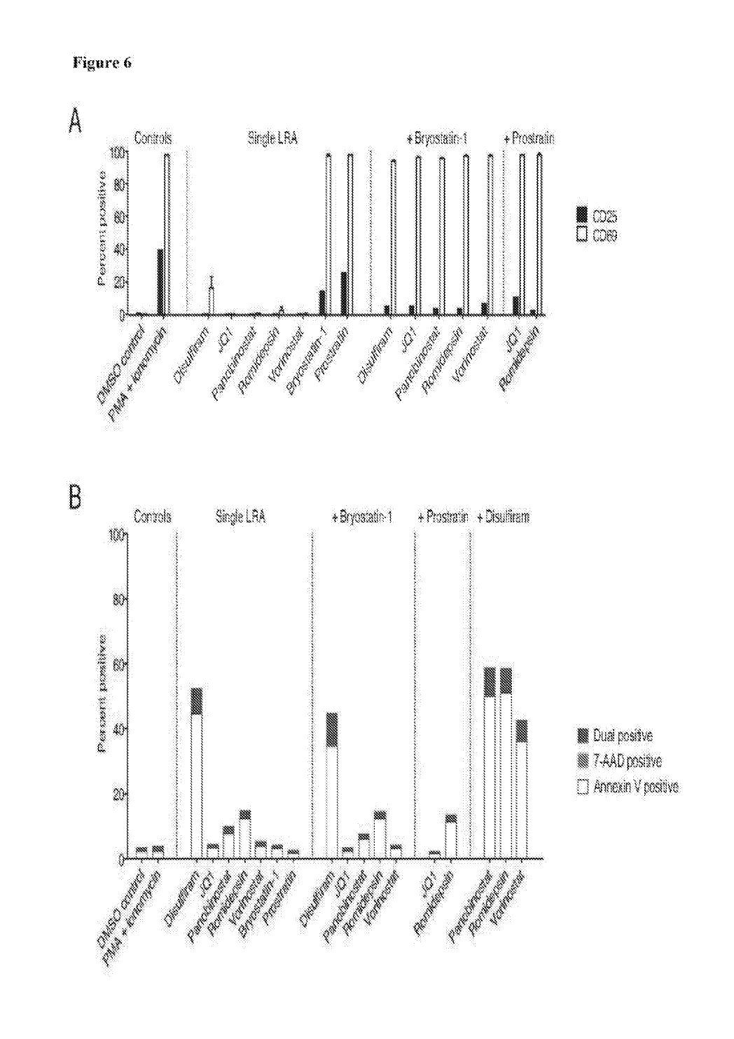

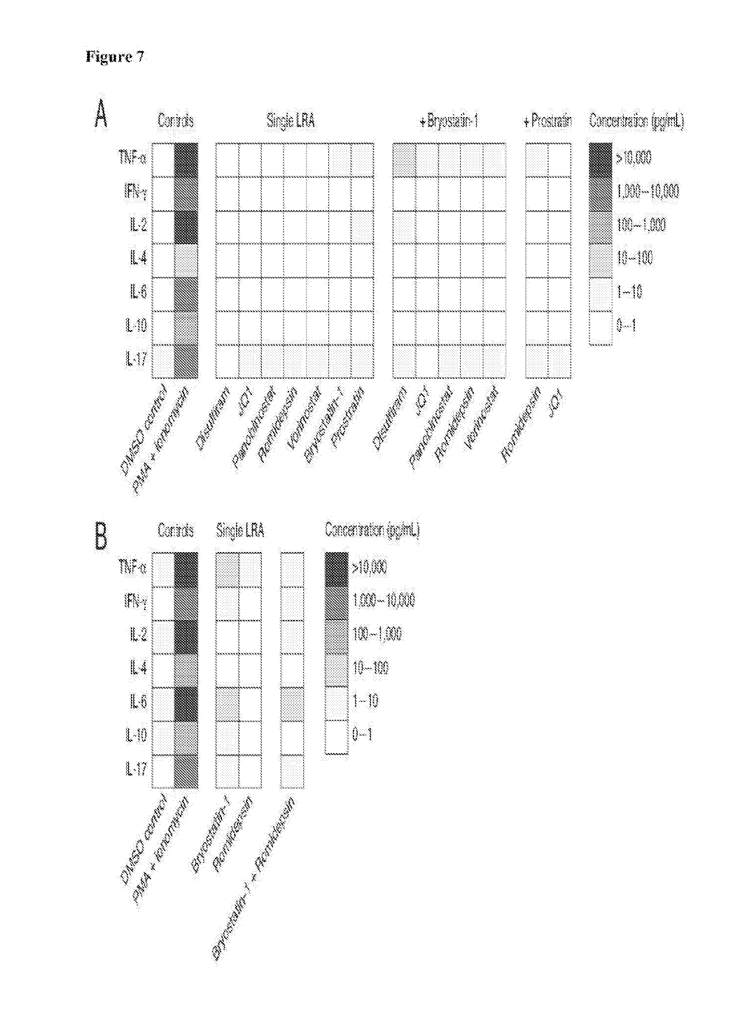

Next it was asked whether robust induction of latent HIV-1 by treatments containing a PKC agonist was coupled with T cell activation or toxicity. rCD4s stimulated with PKC agonists alone or in combination with another LRA exhibited increased surface expression of the early activation marker CD69 (FIG. 6A), consistent with previous studies (14, 48). While some induction of CD25 surface expression on rCD4s occurred after treatment with PKC agonists alone, this expression was reduced with the addition of another LRA (FIG. 6A). Treatments containing a PKC agonist caused minimal decreases in rCD4 cell viability as assessed by annexin V and 7-AAD staining (FIG. 6B). Importantly, combination LRA treatment did not cause cellular toxicity exceeding that caused by single LRA treatment (FIG. 6B). Although activation marker expression is a useful indication of drug activity, the production and release of proinflammatory cytokines provides a more direct measurement of functional T cell activation, especially with regard to potential toxic effects. Global activation by PMA/I treatment induced the production and release of high levels of multiple cytokines from both rCD4s and PBMCs, while treatment with PKC agonists alone or in combination with other LRAs caused little or no cytokine production by rCD4s (FIG. 7A). Similarly, treatment of unfractionated PBMC with PKC agonists alone or in combination with other LRAs caused little or no cytokine production (FIG. 7B).

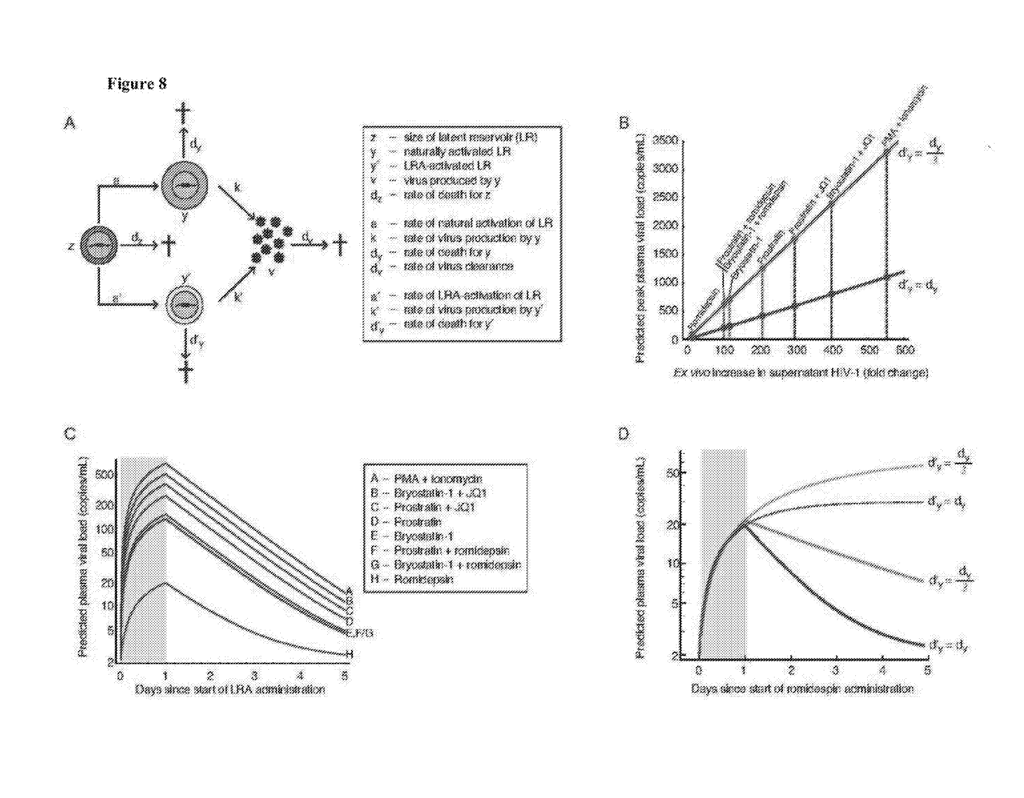

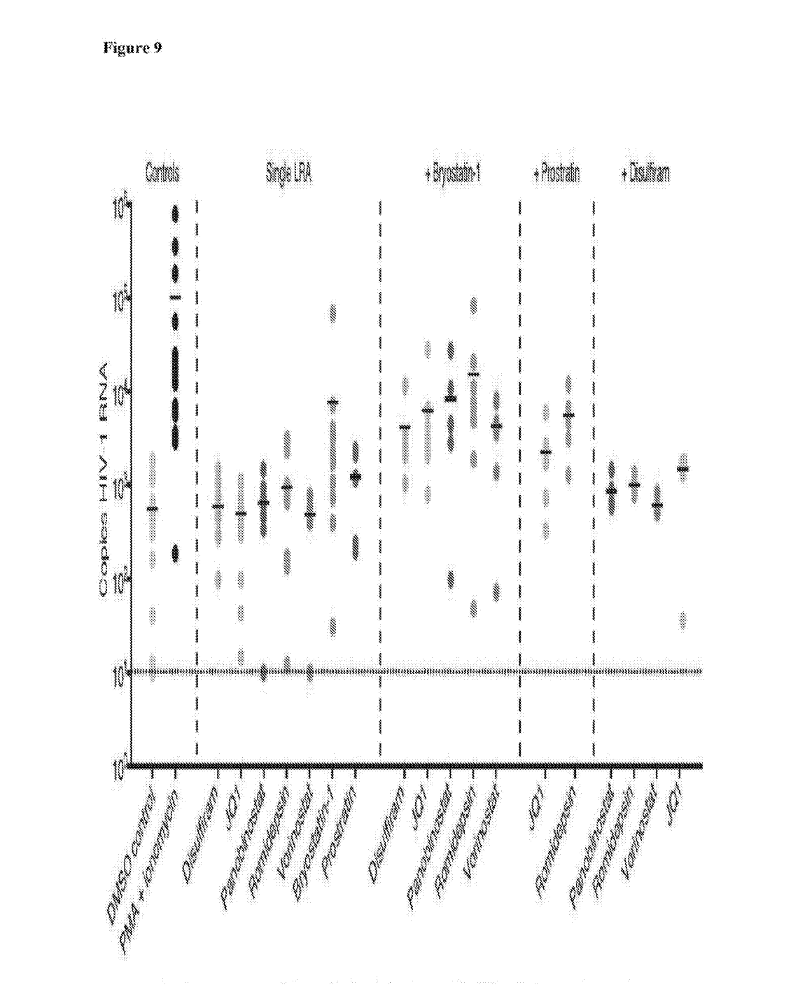

To date, no latency reversing strategy has been shown to reduce the latent reservoir in infected individuals. One potential indication of LRA efficacy in vivo would be a transient increase in plasma HIV-1 RNA levels following LRA administration. To place these results in a broader clinical context, a mathematical model of viral dynamics was used (FIG. 8A; complete description of model in Supplementary Methods) to predict the in vivo changes in plasma HIV-1 levels following LRA treatment from these ex vivo measurements of virus production in response to LRAs. This model assumes that patients are being treated with suppressive ART regimens and have baseline plasma HIV-1 RNA levels below the limit of detection prior to LRA administration. FIG. 8B relates the ex vivo fold-change in supernatant mRNA caused by LRA treatment to the predicted peak plasma HIV-1 RNA that would occur in vivo, if the LRA is administered continuously with activating potential comparable to that in the ex vivo assay, and if the latent reservoir is not replenished by an alternate source (e.g., cryptic viral replication or cellular compartments not affected by the LRA). Combinations including PKC agonists are predicted to cause increases in plasma HIV-1 RNA that are readily measurable with clinical assays (limit of detection of 50 copies/mL). Note that the fold-change for each treatment reported in FIG. 8B is a lower bound for the true value, as no detectable HIV-1 virion production occurred ex vivo for the DMSO control. The actual peak may therefore exceed the prediction shown.

More realistic clinical scenarios involve multiple doses separated by several days or weeks, with each dose active for a short period of time. Under such conditions, the peak plasma HIV-1 RNA level would be expected to decay immediately after LRA activity ceased, and the theoretical peak described in FIG. 8B would not be achieved. In the most conservative scenario considered by this model, LRA-activated cells survive no longer than cells functionally activated by antigenic stimulation, LRA activity lasts for only 24 hours, and no viral replication occurs. Even in this conservative model, plasma HIV-1 RNA levels >100 copies/mL are predicted for all treatments investigated, except for romidepsin (FIG. 8C) which give detectable plasma HIV-1 RNA levels only if LRA-activated cells are assumed to survive three times as long as functionally activated cells (FIG. 8D). Thus, the results predicted by this model are consistent with clinical trials in which HDAC inhibitors alone produce increases in HIV-1 RNA that are close to or below the limit of detection of clinical assays. Fortunately, regimens with stronger latency reversing activity, comparable to the synergistic combinations studied here, should produce readily measurable increases in plasma HIV-1 RNA.

### DISCUSSION

The “shock and kill” strategy for elimination of the HIV-1 latent reservoir in rCD4s requires robust latency reversal. However, given the multifactorial nature of HIV-1 latency, no single drug may be capable of effectively reversing all blocks to proviral gene expression. Indeed, previous studies by the inventors and others have demonstrated that single LRAs are relatively ineffective at reversing latency ex vivo (32-34). These studies suggested that combination therapy comprised of mechanistically distinct LRAs may by required to robustly reverse latency. In this study, two distinct measures of latency reversal were employed to evaluate the efficacy of two-drug LRA combinations in rCD4s from infected individuals.

A number of new two-drug LRA combinations are reported that effectively reverse HIV-1 latency. It was shown that PKC agonists, when combined with JQ1 or a variety of HDAC inhibitors, dramatically induced viral transcription in rCD4s from patients on ART (FIG. 1). This upstream measure of latency reversal revealed drug synergy in these combinations as formally revealed by these analysis based on the Bliss independence model (FIG. 2), which predicts the combined drug effects of drugs with distinct and independent mechanisms. Thus, the finding of synergy for these drug combinations suggests a mechanistically complex interaction. Unraveling the mechanism of these combined effects will further ones' understanding of HIV-1 latency and aid in the design of new LRAs. To this end, a recent study by the Peterlin group suggests that positive transcription elongation factor b (P-TEFb) may play a central role in the combined effects of PKC agonists and HDAC inhibitors in reversing latency (49). PTEF-b, which is required for efficient HIV-1 transcription, is typically present at very low levels in rCD4s. The study by the Peterlin group suggests that the combined effects of PKC agonists and HDAC inhibitors is a result of the induction of PTEF-b production by PKC agonists and the release of this P-TEFb from the inhibitory 7SK-snRNP by HDAC inhibitors. Notably, statistically significant inductions of intracellular HIV-1 mRNA production by when disulfiram was combined with an HDAC inhibitor were observed (FIG. 1). By the rigorous Bliss independence criterion, synergy for the disulfiram combinations tested was not observed (FIG. 2), suggesting that disulfiram and the HDAC inhibitors reverse latency by independent mechanisms. This conclusion is consistent with the proposed mechanisms of latency-reversal by disulfiram (50) and the HDAC inhibitors (49, 51, 52). These findings herein support further study of disulfiram combinations and consideration of future clinical testing.

To extend the assessment of latency reversal, virion release induced by LRA treatment was measured. In this study, treatments including a PKC agonist induced substantial virion release ex vivo, approaching the levels seen with full T cell activation (FIG. 4,5). However, an effective LRA regimen need not induce significant virion production. Viral protein production following latency reversal may be sufficient to drive elimination of these cells by viral cytopathic effects or immune-mediated clearance. Virion production after ex vivo treatment of rCD4s with LRAs was measured, which serves as a proxy for viral protein production. The results suggest that inclusion of PKC agonists in an LRA regimen would be sufficient to induce viral protein production that may lead to the elimination of reactivated cells.

In this study, robust latency reversal in rCD4s from infected individuals with several different combinations of a PKC agonist and an HDAC inhibitor was observed. These results are consistent with a previous report that demonstrated the combined effects of prostratin and vorinostat (37). These findings herein indicate that HDAC inhibitors may be effective as a part of a combination LRA regimen despite relatively limited activity as single agents. Unexpectedly, a recent study demonstrated that certain HDAC inhibitors impair the ability of HIV-1 specific cytotoxic T-lymphocytes (CTL) to kill HIV-1 infected cells, both ex vivo and in in vitro models (53). This impairment of the HIV-1 CTL response by HDAC inhibitors may limit their clinical utility in eradication trials. Importantly, these finding herein that PKC agonists also synergize with JQ1 to robustly reverse latency indicates that HDAC inhibitors are not necessary for robust latency reversal.

These findings herein highlight the potential importance of PKC agonists for latency reversal and provide a rationale for the detailed analysis of the safety profiles of LRA combination therapies containing PKC agonists. While prostratin has not yet been tested in humans, dozens of phase I and phase II clinical trials of bryostatin-1 efficacy in the treatment of a variety of cancers have been safely completed. Lower doses of bryostatin-1 were well tolerated, but dose limiting toxicities grade 3/4 myalgia, arthralgia, and weakness have been observed in patients receiving high doses. While this clinical toxicity has been postulated to result from a cytokine storm induced by bryostatin-1, the induction of proinflammatory cytokine release by PKC agonists at concentrations that effectively reversed HIV-1 latency ex vivo was not observed (FIG. 7). Nevertheless, it is possible that these drugs may have toxic effects unrelated to cytokine production by cells in the peripheral blood. One important question remains: can effective concentrations of bryostatin-1 be achieved in HIV-1 infected individuals? In a recent clinical study of bryostatin-1 in patients with myeloid malignancies, plasma levels of bryostatin-1 were determined using an LC/MS/MS assay in patients receiving bryostatin-1 in combination with GM-CSF (54). Plasma steady state concentrations of bryostatin-1 ranging from roughly 0.2 nM to 1 nM could be achieved in patients receiving the approximated maximally tolerated dose of 16 μg/m2/day continuously infused for 14 or 21 days, and these concentrations could be maintained over the course of the infusion. As presented in FIG. 3, it was found that 1 nM bryostatin-1 induced significant intracellular HIV-1 mRNA production ex vivo when combined with an HDAC inhibitor. Thus, synergies of the kind described here may allow the use of lower, safer doses of PKC agonists. On the basis of the available clinical data and the ex vivo findings, it is suggested that it may be possible to achieve effective concentrations of bryostatin-1 in vivo by taking advantage of synergies of the kind described here. In light of the unpredictable toxicities observed in animal models, such an approach would require extreme caution and very careful patient monitoring. Bryostatin-1 is a natural product available only in small amounts. Several synthetic analogs of both bryostatin-1 and prostratin have recently been developed (48, 55). However, the clinical utility of these analogs remains to be established.

Previous studies of LRAs have given divergent results that can be summarized as follows. Multiple classes of LRAs show high activity in T cell line and primary T cell models of latency. However, each LRA has different levels of activity in different model systems, indicating the need for caution in using these models to define which agents should be advanced into non-human primate studies and clinical trials. Some LRAs also increase HIV-1 RNA production in ex vivo assays using cells from patients on ART (32-34). However, in general, the activity of individual LRAs in these systems is weak compared to maximal T cell activation (32). In clinical trials, HDAC inhibitors have been shown to cause modest increases in cell associated HIV-1 RNA in some studies (28-30, 42, 43), but clear changes in plasma HIV-1 RNA have been seen in only one study to date (43), and no study has demonstrated a decrease in the size of the reservoir or a delay in rebound.

In order to reconcile these diverse outcomes, both increases in intracellular HIV-1 RNA and the production of virus particles following LRA treatment of rCD4 from patients on ART have been measured. Quantitating virus production allowed us to make predictions about how LRA therapy would affect a readily measureable clinical parameter, plasma HIV-1 RNA, using an established model of viral dynamics. Consistent with previous results, individual LRAs induced only minimal increases in cell associated HIV-1 RNA, while substantial increase in HIV-1 RNA were seen with some combinations of LRAs that included a PKC agonist and only treatments including PKC agonists induced significant virus production. The model herein predicts that this level of virus production would result in transient increases in plasma HIV-1 RNA that are readily measurable with standard clinical assays in the context of a clinical trial. However, the predicted levels of HIV-1 induced by single LRAs are generally at or below the detection limit. The estimates generated by this mathematical model are in line with a recently reported clinical trial in which the administration of multiple doses of romidepsin produced detectable plasma HIV-1 RNA levels ranging from 43 to 103 copies/mL in 5 of 6 patients (43). Clinical trials of disulfiram (44), vorinostat (28, 29), and panobinostat (30) found no increases in plasma HIV-1 RNA using quantitative clinical assays, consistent with the observations that these drugs fail to stimulate detectable viral production ex vivo (32) and consistent with the predictions of this mathematical model. As plasma HIV-1 RNA is predicted to change rapidly following LRA administration (FIG. 8C), multiple measurements in the first few hours and days of an LRA trial may be needed to measure latency reversal precisely. This ex vivo analysis of LRA efficacy coupled with modeling of the clinical response to LRA therapy will likely aid in both the selection of candidate LRAs for translation to the clinic and in clinical trial design.

It is cautioned that predicting in vivo viral load changes—a proxy measure for LRA effectiveness—is not the same as predicting the overall decay rate in the latent reservoir over long-term administration. In particular, certain latently infected cells may be resistant to induction by any LRA (56), an effect that is neither measured in these experiments nor included in the model herein. The LRA-induced changes in cell-associated HIV-1 RNA and virion release described here may represent increases in the magnitude of HIV-1 gene expression by a fixed number of cells, increases in the number of cells expressing HIV-1 genes, or a combination of both. The inventors and others are exploring single cell methods to resolve the frequency and amplitude of latency reversal, but with in vivo frequencies on the order of 1 per million, the quantitation of infected cells by such methods is extremely challenging. Flow cytometry based methods are readily applied to primary cell models of HIV-1 latency in which the frequency of latently infected cells is several orders of magnitude higher, and in those models, latency reversing agents clearly increase the number of cells expressing HIV-1 genes (13, 19, 57). Further studies of the fraction of cells induced ex vivo, as well as the lifespan of newly induced cells, may address these questions. It is also important to note that following reversal of latency, infected cells may not die without additional interventions to enhance HIV-1 immunity (58).

There is an increased interest in developing clinical assays that are capable of quantifying the latent reservoir using measures of intracellular or extracellular HIV-1 RNA. However, it is not certain whether either of these HIV-1 RNA measures can be used to accurately measure the frequency of replication competent latent HIV-1 in cells from infected individuals. The data presented in FIG. 5 indicates that intracellular HIV-1 mRNA can be detected in cells that fail to release virions into the supernatant under certain conditions. Recent work by Cillo et al. examined the fraction of proviruses that could be induced by CD3/CD28 co-stimulation to produce intracellular HIV-1 RNA or virions. They found that roughly 7.5% of proviruses produced intracellular HIV-1 RNA while only 1.5% produce virions after co-stimulation. This is consistent with the data as present here (FIG. 5), in which the inventors fail to see a correlation between intracellular and supernatant HIV-1 mRNA measures. While the underlying cause of this discrepancy is not established, these data suggest that intracellular HIV-1 RNA measures may not directly relate to the frequency of replication competent latent HIV-1.

In conclusion, using multiple assays for latency reversal ex vivo in rCD4s from infected individuals, a comparative study to identify highly effective LRA combinations was carried out. Although individual LRAs may cause detectable increases in cell associated HIV-1 RNA, these increases are small in comparison to the effect of T cell activation and are not expected to cause measurable increases in plasma HIV-1 RNA or significant decreases in the latent reservoir. Multiple new two-drug combinations that reverse latency ex vivo were identified. It was demonstrated that PKC agonists combine with JQ1 and with HDAC inhibitors to induce robust reversal of latency to a degree that is comparable to the benchmark of maximal T cell activation. This degree of latency reversal is expected to produce readily measurable transient increases in plasma HIV-1 RNA and hopefully some long-term decrease in the size of the latent reservoir. It was demonstrated that this degree of latency reversal can be achieved without inducing proinflammatory cytokine production, although it remains unclear whether agents like PKC agonists can be safely used in this setting. The methods herein suggest that the experimental and mathematical framework developed here to predict in vivo responses to LRAs will inform the design of future eradication clinical trials.

### Methods:

### Study Subjects

HIV-1-infected individuals were enrolled in the study at Johns Hopkins Hospital based on the criteria of suppressive ART and undetectable plasma HIV-1 RNAs level (<50 copies per mL) for a minimum of 6 months. Characteristics of study participants are presented in Table 1.

### Isolation and Culture of Resting CD4⁺ T Lymphocytes

PBMCs from whole blood or continuous-flow centrifugation leukapheresis product were purified using density centrifugation on a Ficoll-Hypaque gradient. Resting CD4⁺ lymphocytes (CD4+, CD69−, CD25− and HLA-DR−) were enriched by negative depletion as described (32). Cells were cultured in RPMI medium supplemented with 10% fetal bovine serum at a concentration of 5×10⁶ cells per mL for all experiments.

### Latency Reversing Agent Treatment Conditions

Resting CD4⁺ T cells were stimulated with latency reversing agents at the following concentrations for all single and combination treatments unless otherwise indicated: 10 nM bryostatin-1, 300 nM prostratin, 500 nM disulfiram, 1 μM JQ1, 30 nM panobinostat, 40 nM romidepsin, 335 nM vorinostat, 50 ng mL⁻¹ PMA plus 1 μM ionomycin, or media alone plus DMSO. The final DMSO percentage was 0.2% (v/v) for all single and combination treatments. Concentrations were chosen based on previous ex vivo studies with rCD4s from infected individuals as well as studies using in vitro latency models (13, 28, 29, 32-34) with the aim of selecting clinically relevant concentrations.

Measurement of Intracellular HIV-1 mRNA

Five million resting CD4⁺ T cells isolated from HIV-1 infected individuals on suppressive ART were treated with each LRA alone or with the indicated LRA combination in triplicate (single or duplicate if cell number was limiting) for 6 or 24 h in a volume of 1 mL RPMI+10% FBS. Total RNA was isolated, and cDNA synthesis and real-time quantitative PCR were performed as described (32). Briefly, each PCR reaction contained template from approximately one million cell equivalents of cDNA or RNA (for no-RT control reactions). Serial dilutions of a TOPO plasmid containing the last 352 nucleotides of viral genomic RNA plus 30 deoxyadenosines were used for a molecular standard curve. No-RT control reactions were preformed on every treatment sample from only one individual to confirm the absence of signal from contaminating nucleotides but were not done for every individual since the primer/probe set used to detect the 3′ polyadenylated sequence for correctly terminated HIV-1 mRNAs does not amplify HIV-1 proviral DNA (45).

Results from the triplicate samples for each drug treatment were averaged and presented as copies of HIV mRNA per million resting CD4⁺ T cell equivalents, fold change relative to DMSO control, and normalized percentage of the effect of PMA plus ionomycin [(copies_{LRA x}−copies_{DMSO control})/(copies_{PMA+I}−copies_{DMSO control})]. The limit of quantification was 10 copies as described (32). Some samples from one individual yielded a PCR signal of less than 10 copies (undetectable to 9 copies) and were assumed to have 10 copies in calculations of both fold change and normalized percentage of PMA plus ionomycin, and these samples were marked as 10 copies on graphs depicting RNA copies.

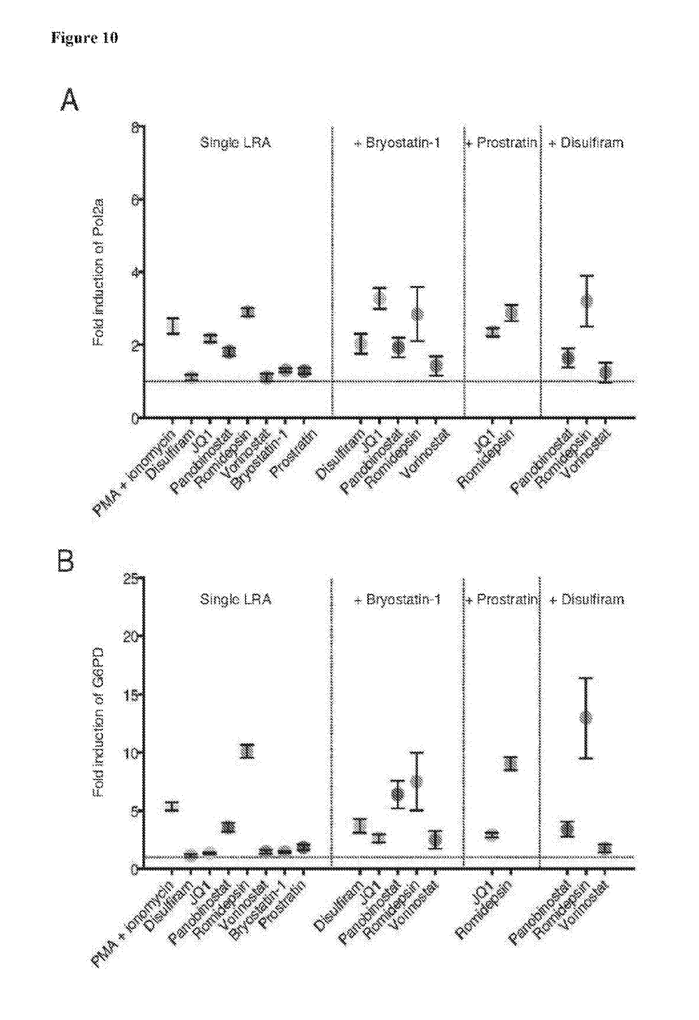

Levels of RNA polymerase II (Pol2) and Glucose-6-phosphate dehydrogenase (G6PD) RNA were also measured for each sample as an endogenous control (TaqMan® Gene Expression Assays Hs00172187_ml and Hs00166169_ml, respectively). The relative fold change for each transcript was determined using the comparative Ct quantification method (relative fold change=2^{-Δct}, ΔCt=Ct_{LRAx}−Ct_{DMSO control}). Particular LRA treatments consistently changed expression of Pol2 and/or G6PD (FIG. 10). Samples treated with the same LRA regiment had near similar levels of Pol2 or G6PD, indicating that the inputs were approximately equal.

Measurement of HIV-1 mRNA in Culture Supernatants

HIV-1 mRNA was extracted from 0.25 mL of supernatant from the LRA-treated cell cultures described above with 0.75 mL of TRIzol® LS Reagent (Invitrogen) according to the manufacturer's protocol. cDNA synthesis and real-time quantitative PCR was performed as described (32). Results were presented as copies of HIV mRNA per mL supernatant and normalized percentage of the effect of PMA plus ionomycin [(copies_{LRA x} copies_{DMSO control})/(copies_{PMA+I}−copies_{DMSO control})]. The limit of detection for each qPCR was 10 copies per reaction, which scaled to a limit of detection of 150 copies per mL of culture supernatant. Primers and probes are listed below. Molecular standard curve was generated as described above.

### Quantitative Analysis of Latency Reversing Agent Combinations

The Bliss independence model, one method to predict the expected combined effects of multiple drugs assuming the drugs act through independent mechanisms, as a metric by which to evaluate the latency reversing activity of drug combinations was used. The Bliss independence model is defined by the equation:

fa_{xy,P}=faₓ+fa_{y}−(faₓ)(fa_{y})

where fa_{xy P} is the predicted fraction affected by a combination of drug X and drug Y given the experimentally observed fraction affected for drug X (faₓ) and drug Y (fa_{y}) individually. The experimentally observed fraction affected by a combination of drug X and drug Y (fa_{xy, o}) can be compared to the predicted fraction affected computed using the Bliss model (fa_{xy, o}) as follows:

Dfa_{xy}=fa_{xy,O}−fa_{xy,P}

If Dfa_{xy}<0 with statistical significance, then the combined effect of the two drugs exceeds that predicted by the Bliss model and the drug combination displays synergy. If Dfa_{xy}=0, then the drug combination follows the Bliss model for independent action. If Dfa_{xy}>0 with statistical significance, then the combined effect of the two drugs is less than that predicted by the Bliss model and the drug combination displays antagonism.
In this analysis, the fraction affected was calculated as follows for intracellular HIV-1 mRNA and for supernatant HIV-1 virion quantitation:

faₓ=(copies drug X−copies DMSO control)/(copies PMA/I−copies DMSO control)

### Flow Cytometry

Resting CD4⁺ T cells isolated from three healthy individuals were incubated with each LRA alone or with the indicated LRA combination in duplicate for 24 hours. The cells were subsequently used to measure the expression levels of T cell activation markers or the frequency of viable cells. For surface receptor analysis, cells were stained with FITC-conjugated anti-human CD69 antibody and PE-conjugated anti-human CD25 antibody (BD Pharmingen). For toxicity analysis, cells were stained for PE-conjugated annexin V and with 7-AAD using the PE Annexin V Apoptosis Detection Kit I (BD Pharmingen). Samples were analyzed using a FACSCalibur flow cytometer and Cell Quest software (Becton Dickinson). Live cell gating in forward versus side scatter plots was performed for T cell activation analysis. Toxicity was defined by the total percentage of Annexin V positivity.

### Cytokine Release Assay

Supernatant was collected from the LRA-treated cell cultures described above and stored at −80° C. for later analysis. Supernatant cytokine levels were determined using Human Th1/Th2/Th17 Cytometric Bead Array (CBA) according to the manufacturer's protocol (BD Biosciences). Briefly, 50 μL supernatant or kit standards were mixed with 50 μL mixed capture beads and 50 μL PE-conjugated detection antibodies and incubated for 3 hours. Then samples were washed to remove unbound PE antibodies and analyzed using a FACSCanto cytometer (BD Biosciences) and FCAP Array software (Soft Flow).

### Primer and Probe Sequences

Nucleotide coordinates are indicated relative to HXB2 consensus sequence. Primers and probe used for HIV mRNA measurement as described (32):

forward (5′→3′)
CAGATGCTGCATATAAGCAGCTG (9501-9523),
reverse (5′→3′)
TTTTTTTTTTTTTTTTTTTTTTTTGAAGCAC (9629-poly A),
probe (5′→3′)
FAM-CCTGTACTGGGTCTCTCTGG-MGB (9531-9550).

### Statistics

Ratio paired Student's t-test was used to determine statistical significance where indicated. A P<0.05 was considered to be statistically significant. Approximately a quarter of the experiments measuring intracellular and supernatant HIV mRNA were blinded. All samples were handled and LRA-treated in the same way for each set of experiments and were not randomized. No statistical method was used to predetermine sample size.

### Supplementary Methods

### 1 Mathematical Model of Viral Dynamics

A system of differential equations was used to describe in vivo viral dynamics during administration of LRA therapy, assuming that co-administered ART suppresses all viral replication. Let z be the abundance of latently infected resting CD4⁺ T cells, let y be the abundance of activated infected CD4⁺ T cells, and let y′ be the abundance of LRA-stimulated infected CD4 T cells that are induced to produce virus, though they may not be functionally activated. Here, activation includes any LRA-independent transition to virus production, such as that caused by stochastic transcriptional changes or by antigenic stimulus. Let v be the plasma viral load, in copies per mL (c mL⁻¹). Since our conclusions will rely only on observed viral load, arbitrary units can be used for the cellular quantities. During fully suppressive ART, viral dynamics can be described by the system,

ż=(a+a′+d_{z})z

\dot{y}=az−d_{y}y

\dot{y}=a′z−d_{y}′y′

\dot{v}=ky+k′y′−dᵥ^{v} (S1)

Here, a and a′ are the rates of activation and LRA-driven induction, respectively. Latently infected cells die at rate d_{z}. To represent the baseline (untreated) rate of reservoir decay due to combined effects of activation and death, we will use the compound parameter δ=a+d_{z}. Activated cells produce virus at rate k and die at rate d_{y}; LRA-induced cells produce virus at rate k′ and die at rate d_{y}′. Since induction is likely not as drastic as functional T cell activation, it is likely for d_{y}′ and k′ to be less than d_{y} and k, respectively [59]. Virus is cleared at rate dᵥ. The values of a′, k′, and d_{y}′ depend on the LRA treatment given. The binary “switch” between latency and (either form of) activity is an idealization; it is possible that transient viral production occurs in cells experiencing varying degrees of latency.

The effect of LRA can be detected by the transient increase in viral load that it causes. To estimate this increase, we rely on observations of the ex vivo system. Specifically, we assume that this system also follows the above viral dynamics, with abundance of extracellular mRNA taking the place of plasma viral load for variable v. We assume moreover that parameter values are the same in vivo as ex vivo, with the exception that dᵥ is zero ex vivo. See Table 1 for discussion of these assumptions.

TABLE 1
Assumptions regarding comparison of in vivo and ex vivo parameters

| | Parameter | | |
|---|---|---|---|
| Parameter | Description | Why assumed same ex & in vivo | Caveats |
| a | Activation rate | Stochastic factors governing viral | Immune-activating effects (MHC class II |
| | | transcription launch a program of viral | presentation, cytokine signaling) not present in |
| | | production [60]; these intracellular | the assay may cause the in vivo value to exceed |
| | | fluctuations may be similar in both settings. | the ex vivo value. |
| a′ | LRA-driven | Assay treatment conditions replicate the in | Immune-activating effects not present in the |
| | induction rate | vivo drug environment. Mechanisms causing | assay may interact with the LRA effect, causing |
| | | induction are believed to rely on the same | the in vivo value to differ from the ex vivo |
| | | cellular transcriptional machinery in both | value. |
| | | settings. | |
| k (k′) | Rate of viral | Viral production occurs intracellularly, and | Cytokine production by CD8⁺ T cells in vivo |
| | production by | primary CD4⁺ T cells studied in the assay | may suppress viral production compared to ex |
| | activated (LRA- | are a close representation of intracellular | vivo rates. |
| | induced) cells | activity in vivo. | |
| d_{y} (d′_{y}) | Death rate of | Production of cytotoxic viral proteins is a | CTL response, not present in the assay, may |
| | activated (LRA- | major cause of cell death and may be similar | alter d_{y} (d′_{y}) in vivo, but see [61, 62] for |
| | induced) cells | in both settings (see parameters k, k′ above). | evidence that this generally is not the case; also |
| | | | see [63] for evidence that HIV-specific |
| | | | responses are generally weak in HIV-infected |
| | | | individuals. |
| d_{z} | Death rate of | Low levels of transcription and viral | Conditions in the assay may not be conducive |
| | latently infected | production in latently infected cells enable | to very long cellular lifespans. Even if this |
| | cells | long cell lifespan ex vivo as in vivo. | parameter differs between the two settings, |
| | | | decay over the short duration of the assay is not |
| | | | expected to have a large effect on observed |
| | | | viral production, as noted in discussion of Eq. |
| | | | ( ). |
| | Parameter | | |
| Parameter | Description | Why assumed zero ex vivo | Caveats |
| dᵥ | Viral decay rate | Viral clearance occurs primarily in | Some decay of viability of virus particles may |
| | | lymphoid and other organs [64]. | also occur over the course of the day-long |
| | | | assay, at a rate slower than in vivo. |

Generally, we assume d_{y}≥d_{y}′ and that both of these cell death rates are much larger than, a, a′, and d_{z}. Below we state explicitly where these assumptions are used.

### 2 Analysis of Ex Vivo Dynamics

Following the above discussion, we assume dᵥ=0. The assay begins with only resting CD4⁺ T cells, implying initial condition v(0)=y(0)=y′(0)=0 and z(0)=z₀, where z₀ is the number of latently infected cells collected from the cell donor (a small fraction of the 5 million cells). Since dᵥ=0, the virus simply accumulates over time. The DMSO control provides no inducing effect beyond the baseline rate a, and the solution of system ( ) for this case is

$${v}_{DMSO} ( {t}_{a} ) = \frac{{akz}_{0}}{{d}_{y} δ ( {d}_{y} - δ )} ( {d}_{y} ( 1 - {e}^{- δ {t}_{a}} ) - δ ( 1 - {e}^{- {d}_{y} {t}_{a}} ) ) , \qquad ( S2 )$$

where the subscript in tₐ indicates time in the assay, which will later be distinguished from time in vivo. Adding treatment applies a non-zero a′. The amount of extracellular mRNA is therefore increased by a factor:

$$\frac{{v}_{LRA} ( {t}_{a} )}{{v}_{DMSO} ( {t}_{a} )} \approx 1 + \frac{{a}^{′} {k}^{′}}{ak} ( 1 + \frac{{t}_{a}}{3} ( {d}_{y} - {d}_{y}^{′} - {a}^{′} - \frac{ak}{{k}^{′}} ) ) . \qquad ( S3 )$$

This approximation holds for δtₐ«1 and (tₐ/3)(d_{y}−d_{y}′−a′−ak/k′) near to or less than one; both are expected as tₐ≤1 day in the assay, δ is the slow rate of reservoir decay (half-life of many months), and the other rate parameters are no more than 1 day⁻¹. Let ρ be the observed value of v_{LRA}(tₐ)v_{DMSO}(tₐ) at the end of the assay. From this observation, we can estimate the following parameter ratio:

$$\frac{{k}^{′}}{k} \approx ( \frac{a}{{a}^{′}} ) ( \frac{3 ( ρ - 1 ) {t}_{a} {a}^{′}}{3 - {t}_{a} ( {a}^{′} + {d}_{y} - {d}_{y}^{′} )} ) . \qquad ( S4 )$$

This parameter ratio estimate is used to predict viral load in vivo, below.

### 3 Analysis of In Vivo Dynamics

Since virus is subject to rapid decay in vivo, we can treat it using the commonly used quasi-steady state approximation: v(t)=(ky(t)+k′y′(t))/dᵥ [65]. Likewise, since death rate d_{y} greatly exceeds baseline activation rate a, the initial number of actively infected cells can be approximated by activation-death equilibrium, y(0)=az₀/d_{y}, implying a residual viral load of v(0)=akz₀/(d_{y}dᵥ). The fractional increase in viral load caused by administering the LRA for a period of time t follows from these assumptions and system ( ):

$$\frac{{v}_{LRA} ( t )}{v ( 0 )} = \frac{{d}_{y} {e}^{- ( δ + {a}^{′} ) t} - ( δ + {a}^{′} ) {e}^{- {d}_{y} t}}{{d}_{y} - δ - {a}^{′}} + \frac{{d}_{y} ( z ( ρ - ) + {a}^{′} {t}_{a} ) ( {e}^{- ( δ + {a}^{′} ) t} - {e}^{- {d}_{y}^{′} t} )}{( {d}_{y}^{′} - δ - {a}^{′} ) ( 3 + {t}_{a} ( {d}_{y} - {d}_{y}^{′} - {a}^{′} ) )} . \qquad ( S5 )$$

Here, eq. ( ) has been used to eliminate both k and k′ by introducing the ex vivo-observed parameter ρ. The first line of ( ) represents viremia due to activated cells, while the second line represents viremia due to LRA-induced cells.

The in vivo viral load ratio in ( ) approximates a bi-exponential curve, initially rising linearly from 1 at rate ≈d_{y}(ρ−1) and ultimately decaying exponentially at rate δ+a′. The maximum value cannot be expressed in a simple form, but the peak viral load ratio can be approximated by noting that the first line of ( ) falls between 0 and 1, while the second line (for which the maximum can be expressed in closed form) has a peak much larger than 1 for typical parameter values (d_{y}≥d_{y}′>a′>δ, none of these rates much larger than 1 day⁻¹, and ρ»1). The peak viral load, relative to the baseline residual viral load, is therefore approximately

$$\frac{max ( {v}_{LRA} ( t ) )}{v ( 0 )} {\approx }^{<} 1 + ( \frac{{d}_{y}}{{d}_{y}^{′}} ) ( 3 ( ρ - 1 ) + {a}^{′} {t}_{a} ) ( \frac{{( ( δ + {a}^{′} ) \text{/} {d}_{y}^{′} )}^{( ( δ + {a}^{′} ) \text{/} ( {d}_{y}^{′} - δ - {a}^{′} ) )}}{3 + {t}_{a} ( {d}_{y} - {d}_{y}^{′} - {a}^{′} )} ) , \qquad ( S6 )$$

and it occurs approximately at time

$${t}_{max} \approx \frac{ln ( {d}_{y}^{′} \text{/} ( δ + {a}^{′} ) )}{{d}_{y}^{′} - δ - {a}^{′}} . \qquad ( S7 )$$

The approximation in Eq. ( ) never overestimates the true peak ratio by more than 1. Note that the exponentiated expression decreases with the sum (δ+a′), indicating the effect of a rapidly decaying reservoir on the peak viral load. If (δ+a′) is very small relative to d_{y}′, then the peak viral load is simply

$$\frac{max ( {v}_{LRA} ( t ) )}{v ( 0 )} {\approx }^{<} 1 + ( ρ - 1 ) \frac{{d}_{y}}{{d}_{y}^{′}} . \qquad ( S8 )$$

This approximation is used in FIG. 7B. Note that this result does not depend on the LRA-driven induction rate a′ nor the viral production rate k′ of LRA-induced cells; the experimentally observed parameter ρ depends on a combination of induction and production. Further experiments—involving measurement of the fraction of cells induced or the decay in viral production over time—would be needed to resolve rate a′, which determines the rate at which LRA therapy would ultimately deplete the latent reservoir.

### 4 In Vivo Dynamics for Short Treatment Window

The previous section assumes that treatment is administered continuously, until the latent reservoir eventually decays completely, yet such a regimen may not be achievable. Suppose instead that the effect of treatment ceases at time t_{Stop}, after which point a′ is set to zero. For t>tₛₜₒₚ, the viral load ratio is:

$$\frac{{v}_{LRA} ( t )}{v ( 0 )} = \frac{{e}^{- ( {d}_{y} + δ ) t″ ( δ + {a}^{′} ) {t}_{Stop}}}{( {d}_{y} - δ ) ( {d}_{y} - δ - {a}^{′} )} \times [ {d}_{y}^{2} {e}^{{d}_{y} t + δ {t}_{Stop}} - {d}_{y} ( ( δ + {a}^{′} ) ( {e}^{{d}_{y} t + δ {t}_{Stop}} + {e}^{δ t + ( δ + {a}^{′} ) {t}_{Stop}} ) - {a}^{′} {e}^{δ t + {d}_{y} {t}_{Stop}} ) + δ ( δ + {a}^{′} ) {e}^{δ t + ( δ + {a}^{′} ) {t}_{Stop}} ] + {e}^{- {d}_{y}^{′} ( t - {t}_{Stop} )} \frac{{d}_{y} ( 3 ( ρ - 1 ) + {a}^{′} {t}_{a} ) ( {e}^{- ( δ + {a}^{′} ) {t}_{Stop}} - {e}^{- {d}_{y}^{′} {t}_{Stop}} )}{( {d}_{y}^{′} - δ - {a}^{′} ) ( 3 + {t}_{a} ( {d}_{y} - {a}^{′} - {d}_{y}^{′} ) )} . \qquad ( S9 )$$

As in Eq. ( ) the first term (spanning the first three lines) represents the portion due to activated cells, while the second term (on the final line) represents the portion due to LRA-induced cells. This expression is used to compute the curves in FIGS. 7C and 7D. Note that this dynamic treats the LRA as pharmacologically active at maximum concentration at the start of therapy; a more realistic model would include an absorption phase during which viral load may increase more gradually.

### 5 Parameters Used in FIG. 7

For each treatment described in FIG. 7B, ρ was chosen to match the median value observed in the ex vivo assay, given in Table 2. To provide viral load estimates, pre-treatment residual viremia of 2 c m1⁻¹ was used. Eq. ( ) was used to compute peak viral load, with d_{y}/d_{y}′ of 1 or 3.

For FIGS. 7C and 7D, Eq. ( ) was used, and both ρ and pre-treatment residual viremia were as in FIG. 7B. Baseline activation rate a=5.7×10⁻⁵ day⁻¹ and latent cell death rate d_{z}=4.66×10⁻⁴ day⁻¹ were chosen to be consistent with observed residual viremia and reservoir half-life of 44 months [66]. Death rate d_{y} was set to 1 day⁻¹ [67], and d_{y}′ was either 1 day⁻¹ (blue curves) or 1/3 days⁻¹ (red curves). For blue curves, a′ for each treatment was chosen using the relationship ( ), assuming d_{y}/d_{y}′=k/k′=1 (see Table 2). For red curves displaying romidepsin treatment, a′ of 0.002 day⁻¹ was chosen to be consistent with d_{y}/d_{y} ′=3.

TABLE 2
Treatment-specific parameters used
for blue curves in FIGS. 7C and 7D

| Treatment | ρ | a′ (day⁻¹) |
|---|---|---|
| Romidepsin | 15 | 8 × 10⁻⁴ |
| Prostratin + romidepsin | 104 | 0.0059 |
| Bryostatin-1 + romidepsin | 105 | 0.0059 |
| Bryostatin-1 | 120 | 0.0068 |
| Prostratin | 209 | 0.012 |
| Prostratin + JQ1 | 297 | 0.017 |
| Bryostatin-1 + JQ1 | 401 | 0.023 |
| PMA + ionomycin | 554 | 0.032 |

### Study Approval

The Johns Hopkins Institutional Review granted approval for this study. All research participants enrolled in this study provided written informed consent prior to inclusion in this study.

### REFERENCES

1. Chun, T. W., Finzi, D., Margolick, J., Chadwick, K., Schwartz, D., and Siliciano, R. F. 1995. In vivo fate of HIV-1-infected T cells: quantitative analysis of the transition to stable latency. Nat Med 1:1284-1290.
2. Chun, T. W., Carruth, L., Finzi, D., Shen, X., DiGiuseppe, J. A., Taylor, H., Hermankova, M., Chadwick, K., Margolick, J., Quinn, T. C., et al. 1997. Quantification of latent tissue reservoirs and total body viral load in HIV-1 infection. Nature 387:183-188.
3. Chun, T. W., Stuyver, L., Mizell, S. B., Ehler, L. A., Mican, J. A., Baseler, M., Lloyd, A. L., Nowak, M. A., and Fauci, A. S. 1997. Presence of an inducible HIV-1 latent reservoir during highly active antiretroviral therapy. Proc Natl Acad Sci USA 94:13193-13197.
4. Finzi, D., Hermankova, M., Pierson, T., Carruth, L. M., Buck, C., Chaisson, R. E., Quinn, T. C., Chadwick, K., Margolick, J., Brookmeyer, R., et al. 1997. Identification of a reservoir for HIV-1 in patients on highly active antiretroviral therapy. Science 278:1295-1300.
5. Wong, J. K., Hezareh, M., Gunthard, H. F., Havlir, D. V., Ignacio, C. C., Spina, C. A., and Richman, D. D. 1997. Recovery of replication-competent HIV despite prolonged suppression of plasma viremia. Science 278:1291-1295.
6. Hermankova, M., Siliciano, J. D., Zhou, Y., Monie, D., Chadwick, K., Margolick, J. B., Quinn, T. C., and Siliciano, R. F. 2003. Analysis of human immunodeficiency virus type 1 gene expression in latently infected resting CD4+ T lymphocytes in vivo. J Virol 77:7383-7392.
7. Finzi, D., Blankson, J., Siliciano, J. D., Margolick, J. B., Chadwick, K., Pierson, T., Smith, K., Lisziewicz, J., Lori, F., Flexner, C., et al. 1999. Latent infection of CD4+ T cells provides a mechanism for lifelong persistence of HIV-1, even in patients on effective combination therapy. Nat Med 5:512-517.
8. Siliciano, J. D., Kajdas, J., Finzi, D., Quinn, T. C., Chadwick, K., Margolick, J. B., Kovacs, C., Gange, S. J., and Siliciano, R. F. 2003. Long-term follow-up studies confirm the stability of the latent reservoir for HIV-1 in resting CD4+ T cells. Nat Med 9:727-728.
9. Strain, M. C., Gunthard, H. F., Havlir, D. V., Ignacio, C. C., Smith, D. M., Leigh-Brown, A. J., Macaranas, T. R., Lam, R. Y., Daly, O. A., Fischer, M., et al. 2003. Heterogeneous clearance rates of long-lived lymphocytes infected with HIV: intrinsic stability predicts lifelong persistence. Proc Natl Acad Sci USA 100:4819-4824.
10. Deeks, S. G. 2012. HIV: Shock and kill. Nature 487:439-440.
11. Prins, J. M., Jurriaans, S., van Praag, R. M., Blaak, H., van Rij, R., Schellekens, P. T., ten Berge, I. J., Yong, S. L., Fox, C. H., Roos, M. T., et al. 1999. Immuno-activation with anti-CD3 and recombinant human IL-2 in HIV-1-infected patients on potent antiretroviral therapy. AIDS 13:2405-2410.
12. Xing, S., and Siliciano, R. F. 2012. Targeting HIV latency: pharmacologic strategies toward eradication. Drug Discov Today.
13. Spina, C. A., Anderson, J., Archin, N. M., Bosque, A., Chan, J., Famiglietti, M., Greene, W. C., Kashuba, A., Lewin, S. R., Margolis, D. M., et al. 2013. An in-depth comparison of latent HIV-1 reactivation in multiple cell model systems and resting CD4+ T cells from aviremic patients. PLoS Pathog 9:e1003834.
14. Korin, Y. D., Brooks, D. G., Brown, S., Korotzer, A., and Zack, J. A. 2002. Effects of prostratin on T-cell activation and human immunodeficiency virus latency. J Virol 76:8118-8123.
15. Williams, S. A., Chen, L. F., Kwon, H., Fenard, D., Bisgrove, D., Verdin, E., and Greene, W. C. 2004. Prostratin antagonizes HIV latency by activating NF-kappaB. J Biol Chem 279:42008-42017.
16. Choudhary, S. K., Archin, N. M., and Margolis, D. M. 2008. Hexamethylbisacetamide and disruption of human immunodeficiency virus type 1 latency in CD4(+) T cells. J Infect Dis 197:1162-1170.
17. Archin, N. M., Espeseth, A., Parker, D., Cheema, M., Hazuda, D., and Margolis, D. M. 2009. Expression of latent HIV induced by the potent HDAC inhibitor suberoylanilide hydroxamic acid. AIDS Res Hum Retroviruses 25:207-212.
18. Contreras, X., Schweneker, M., Chen, C. S., McCune, J. M., Deeks, S. G., Martin, J., and Peterlin, B. M. 2009. Suberoylanilide hydroxamic acid reactivates HIV from latently infected cells. J Biol Chem 284:6782-6789.
19. Yang, H. C., Xing, S., Shan, L., O'Connell, K., Dinoso, J., Shen, A., Zhou, Y., Shrum, C. K., Han, Y., Liu, J. O., et al. 2009. Small-molecule screening using a human primary cell model of HIV latency identifies compounds that reverse latency without cellular activation. J Clin Invest 119:3473-3486.
20. Friedman, J., Cho, W. K., Chu, C. K., Keedy, K. S., Archin, N. M., Margolis, D. M., and Karn, J. 2011. Epigenetic silencing of HIV-1 by the histone H3 lysine 27 methyltransferase enhancer of Zeste 2. J Virol 85:9078-9089.
21. Xing, S., Bullen, C. K., Shroff, N. S., Shan, L., Yang, H. C., Manucci, J. L., Bhat, S., Zhang, H., Margolick, J. B., Quinn, T. C., et al. 2011. Disulfiram reactivates latent HIV-1 in a Bcl-2-transduced primary CD4+ T cell model without inducing global T cell activation. J Virol 85:6060-6064.
22. Banerjee, C., Archin, N., Michaels, D., Belkina, A. C., Denis, G. V., Bradner, J., Sebastiani, P., Margolis, D. M., and Montano, M. 2012. BET bromodomain inhibition as a novel strategy for reactivation of HIV-1. J Leukoc Biol 92:1147-1154.
23. Gallastegui, E., Marshall, B., Vidal, D., Sanchez-Duffhues, G., Collado, J. A., Alvarez-Fernandez, C., Luque, N., Terme, J. M., Gatell, J. M., Sanchez-Palomino, S., et al. 2012. Combination of biological screening in a cellular model of viral latency and virtual screening identifies novel compounds that reactivate HIV-1. J Virol 86:3795-3808.
24. Xing, S., Bhat, S., Shroff, N. S., Zhang, H., Lopez, J. A., Margolick, J. B., Liu, J. O., and Siliciano, R. F. 2012. Novel structurally related compounds reactivate latent HIV-1 in a bcl-2-transduced primary CD4+ T cell model without inducing global T cell activation. J Antimicrob Chemother 67:398-403.
25. Boehm, D., Calvanese, V., Dar, R. D., Xing, S., Schroeder, S., Martins, L., Aull, K., Li, P. C., Planelles, V., Bradner, J. E., et al. 2013. BET bromodomain-targeting compounds reactivate HIV from latency via a Tat-independent mechanism. Cell Cycle 12:452-462.
26. Novis, C. L., Archin, N. M., Buzon, M. J., Verdin, E., Round, J. L., Lichterfeld, M., Margolis, D. M., Planelles, V., and Bosque, A. 2013. Reactivation of latent HIV-1 in central memory CD4(+) T cells through TLR-1/2 stimulation. Retrovirology 10:119.
27. Dar, R. D., Hosmane, N. N., Arkin, M. R., Siliciano, R. F., and Weinberger, L. S. 2014. Screening for noise in gene expression identifies drug synergies. Science 344:1392-1396.
28. Archin, N. M., Bateson, R., Tripathy, M., Crooks, A. M., Yang, K. H., Dahl, N. P., Kearney, M. F., Anderson, E. M., Coffin, J. M., Strain, M. C., et al. 2014. HIV-1 Expression within Resting CD4 T-Cells Following Multiple Doses of Vorinostat. J Infect Dis.
29. Archin, N. M., Liberty, A. L., Kashuba, A. D., Choudhary, S. K., Kuruc, J. D., Crooks, A. M., Parker, D. C., Anderson, E. M., Kearney, M. F., Strain, M. C., et al. 2012. Administration of vorinostat disrupts HIV-1 latency in patients on antiretroviral therapy. Nature 487:482-485.
30. Rasmussen, T. A., Tolstrup, M., Brinkmann, C. R., Olesen, R., Erikstrup, C., Solomon, A., Winckelmann, A., Palmer, S., Dinarello, C., Buzon, M., et al. 2014. Panobinostat, a histone deacetylase inhibitor, for latent-virus reactivation in HIV-infected patients on suppressive antiretroviral therapy: a phase 1/2, single group, clinical trial. The Lancet HIV 1:e13-e21.
31. Blazkova, J., Chun, T. W., Belay, B. W., Murray, D., Justement, J. S., Funk, E. K., Nelson, A., Hallahan, C. W., Moir, S., Wender, P. A., et al. 2012. Effect of histone deacetylase inhibitors on HIV production in latently infected, resting CD4(+) T cells from infected individuals receiving effective antiretroviral therapy. J Infect Dis 206:765-769.
32. Bullen, C. K., Laird, G. M., Durand, C. M., Siliciano, J. D., and Siliciano, R. F. 2014. New ex vivo approaches distinguish effective and ineffective single agents for reversing HIV-1 latency in vivo. Nat Med.
33. Cillo, A. R., Sobolewski, M. D., Bosch, R. J., Fyne, E., Piatak, M., Jr., Coffin, J. M., and Mellors, J. W. 2014. Quantification of HIV-1 latency reversal in resting CD4+ T cells from patients on suppressive antiretroviral therapy. Proc Natl Acad Sci USA.
34. Wei, D. G., Chiang, V., Fyne, E., Balakrishnan, M., Barnes, T., Graupe, M., Hesselgesser, J., Irrinki, A., Murry, J. P., Stepan, G., et al. 2014. Histone Deacetylase Inhibitor Romidepsin Induces HIV Expression in CD4 T Cells from Patients on Suppressive Antiretroviral Therapy at Concentrations Achieved by Clinical Dosing. PLoS Pathog 10:e1004071.
35. Laird, G. M., Eisele, E. E., Rabi, S. A., Lai, J., Chioma, S., Blankson, J. N., Siliciano, J. D., and Siliciano, R. F. 2013. Rapid Quantification of the Latent Reservoir for HIV-1 Using a Viral Outgrowth Assay. PLoS Pathog 9:e1003398.
36. Williams, S. A., Chen, L. F., Kwon, H., Ruiz-Jarabo, C. M., Verdin, E., and Greene, W. C. 2006. NF-kappaB p50 promotes HIV latency through HDAC recruitment and repression of transcriptional initiation. EMBO J 25:139-149.
37. Reuse, S., Calao, M., Kabeya, K., Guiguen, A., Gatot, J. S., Quivy, V., Vanhulle, C., Lamine, A., Vaira, D., Demonte, D., et al. 2009. Synergistic activation of HIV-1 expression by deacetylase inhibitors and prostratin: implications for treatment of latent infection. PLoS One 4:e6093.
38. Kauder, S. E., Bosque, A., Lindqvist, A., Planelles, V., and Verdin, E. 2009. Epigenetic regulation of HIV-1 latency by cytosine methylation. PLoS Pathog 5:e1000495.
39. Burnett, J. C., Lim, K. I., Calafi, A., Rossi, J. J., Schaffer, D. V., and Arkin, A. P. 2010. Combinatorial latency reactivation for HIV-1 subtypes and variants. J Virol 84:5958-5974.
40. Mbonye, U., and Karn, J. 2014. Transcriptional control of HIV latency: cellular signaling pathways, epigenetics, happenstance and the hope for a cure. Virology 454-455:328-339.
41. Bouchat, S., Gatot, J. S., Kabeya, K., Cardona, C., Colin, L., Herbein, G., De Wit, S., Clumeck, N., Lambotte, O., Rouzioux, C., et al. 2012. Histone methyltransferase inhibitors induce HIV-1 recovery in resting CD4(+) T cells from HIV-1-infected HAART-treated patients. AIDS 26:1473-1482.
42. Elliott, J. H., Wightman, F., Solomon, A., Ghneim, K., Ahlers, J., Cameron, M. J., Smith, M. Z., Spelman, T., McMahon, J., Velayudham, P., et al. 2014. Activation of HIV Transcription with Short-Course Vorinostat in HIV-Infected Patients on Suppressive Antiretroviral Therapy. PLoS Pathog 10:e1004473.
43. Søgaard, O. S., Graversen, M. E., Leth, S., Brinkmann, C. R., Kær, A. S., Olesen, R., Denton, P. W., Nissen, P., Sommerfelt, M., Rasmussen, T. A., et al. 2014. The HDAC inhibitor romidepsin is safe and effectively reverses HIV-1 latency in vivo as measured by standard clinical assays. paper presented at International AIDS Society 20th International AIDS conference.
44. Spivak, A. M., Andrade, A., Eisele, E., Hoh, R., Bacchetti, P., Bumpus, N. N., Emad, F., Buckheit, R., 3rd, McCance-Katz, E. F., Lai, J., et al. 2014. A Pilot Study Assessing the Safety and Latency-Reversing Activity of Disulfiram in HIV-1-Infected Adults on Antiretroviral Therapy. Clin Infect Dis 58:883-890.
45. Shan, L., Rabi, S. A., Laird, G. M., Eisele, E., Zhang, H., Margolick, J. B., and Siliciano, R. F. 2013. A novel PCR assay for quantification of HIV-1 RNA. J Virol.
46. Chou, T. C. 2006. Theoretical basis, experimental design, and computerized simulation of synergism and antagonism in drug combination studies. Pharmacol Rev 58:621-681.
47. Bliss, C. I. 1939. The toxicity of poisons applied jointly. Annals of Applied Biology 26:585-615.
48. DeChristopher, B. A., Loy, B. A., Marsden, M. D., Schrier, A. J., Zack, J. A., and Wender, P. A. 2012. Designed, synthetically accessible bryostatin analogues potently induce activation of latent HIV reservoirs in vitro. Nat Chem 4:705-710.
49. Bartholomeeusen, K., Fujinaga, K., Xiang, Y., and Peterlin, B. M. 2013. Histone Deacetylase Inhibitors (HDACis) That Release the Positive Transcription Elongation Factor b (P-TEFb) from Its Inhibitory Complex Also Activate HIV Transcription. J Biol Chem 288:14400-14407.
50. Doyon, G., Zerbato, J., Mellors, J. W., and Sluis-Cremer, N. 2013. Disulfiram reactivates latent HIV-1 expression through depletion of the phosphatase and tensin homolog. AIDS 27:F7-F11.
51. Archin, N. M., Keedy, K. S., Espeseth, A., Dang, H., Hazuda, D. J., and Margolis, D. M. 2009. Expression of latent human immunodeficiency type 1 is induced by novel and selective histone deacetylase inhibitors. AIDS 23:1799-1806.
52. Keedy, K. S., Archin, N. M., Gates, A. T., Espeseth, A., Hazuda, D. J., and Margolis, D. M. 2009. A limited group of class I histone deacetylases acts to repress human immunodeficiency virus type 1 expression. J Virol 83:4749-4756.
53. Jones, R. B., O'Connor, R., Mueller, S., Foley, M., Szeto, G. L., Karel, D., Lichterfeld, M., Kovacs, C., Ostrowski, M. A., Trocha, A., et al. 2014. Histone deacetylase inhibitors impair the elimination of HIV-infected cells by cytotoxic T-lymphocytes. PLoS Pathog 10:e1004287.
54. Smith, B. D., Jones, R. J., Cho, E., Kowalski, J., Karp, J. E., Gore, S. D., Vala, M., Meade, B., Baker, S. D., Zhao, M., et al. 2011. Differentiation therapy in poor risk myeloid malignancies: Results of a dose finding study of the combination bryostatin-1 and GM-CSF. Leuk Res 35:87-94.
55. Beans, E. J., Fournogerakis, D., Gauntlett, C., Heumann, L. V., Kramer, R., Marsden, M. D., Murray, D., Chun, T. W., Zack, J. A., and Wender, P. A. 2013. Highly potent, synthetically accessible prostratin analogs induce latent HIV expression in vitro and ex vivo. Proc Natl Acad Sci USA 110:11698-11703.
56. Ho, Y. C., Shan, L., Hosmane, N. N., Wang, J., Laskey, S. B., Rosenbloom, D. I., Lai,
J., Blankson, J. N., Siliciano, J. D., and Siliciano, R. F. 2013. Replication-competent noninduced proviruses in the latent reservoir increase barrier to HIV-1 cure. Cell 155:540-551.
57. Bosque, A., and Planelles, V. 2009. Induction of HIV-1 latency and reactivation in primary memory CD4+ T cells. Blood 113:58-65.
58. Shan, L., Deng, K., Shroff, N. S., Durand, C. M., Rabi, S. A., Yang, H. C., Zhang, H., Margolick, J. B., Blankson, J. N., and Siliciano, R. F. 2012. Stimulation of HIV-1-specific cytolytic T lymphocytes facilitates elimination of latent viral reservoir after virus reactivation. Immunity 36:491-501.
59. L. Shan, et al., J Antimicrob. Chemother. 69, 28 (2014).
60. A. Singh, L. S. Weinberger, Curr. Opin. Microbiol. 12, 460 (2009).
61. N. R. Klatt, et al., PLoS Pathog. 6, e1000747 (2010).
62. J. K. Wong, et al., PLoS Pathog. 6, e1000748 (2010).
63. L. Shan, et al., Immunity 36, 491 (2012).
64. L. Zhang, P. J. Dailey, A. Gettie, J. Blanchard, D. D. Ho, J Virol. 76, 5271 (2002).
65. A. S. Perelson, A. U. Neumann, M. Markowitz, J. M. Leonard, D. D. Ho, Science 271, 1582 (1996).
66. A. L. Hill, D. I. S. Rosenbloom, F. Fu, M. A. Nowak, R. F. Siliciano, Proc. Natl. Acad. Sci. USA 111, 13475 (2014).
67. M. Markowitz, et al., J Virol. 77, 5037 (2003).

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

1. A method of preventing or treating a HIV infection comprising administering to a mammal in need thereof, a therapeutically effective amount of a composition comprising combinations of a latency reversing agent.

2. The method of claim 1, wherein the combination comprises a PKC (protein kinase c) agonist, NFκB (nuclear factor kappa-light-chain-enhancer of activated B cells) or AKT pathway activator, a HDAC (histone deacetylase) inhibitor, bromodomain inhibitor, or combination thereof.

3. The method of claim 2, wherein the PKC agonist is bryostatin-1 or prostratin, the NFκB or AKT pathway activator is disulfiram, the HDAC inhibitor is vorinostat, panobinostat, or romidepsin, or the bromodomain inhibitor is JQ1.

4. (canceled)

5. (canceled)

6. (canceled)

7. The method of claim 2, wherein the combination comprises the NFκB or AKT pathway activator in combination with the HDAC inhibitor, the PKC agonist in combination with the bromodomain inhibitor, or the PKC agonist in combination with the HDAC inhibitor.

8. The method of claim 7, wherein the NFκB or AKT pathway activator is disulfiram and the HDAC inhibitor is vorinostat, the NFκB or AKT pathway activator is disulfiram and the HDAC inhibitor is panobinostat, or the NFκB or AKT pathway activator is disulfiram and the HDAC inhibitor is romidepsin.

9. (canceled)

10. (canceled)

11. (canceled)

12. The method of claim 7, wherein the PKC agonist is bryostatin-1 and the bromodomain inhibitor is JQ1 or the PKC agonist is prostratin and the bromodomain inhibitor is JQ1.

13. (canceled)

14. (canceled)

15. The method of claim 7, wherein the PKC agonist is bryostatin-1 and the HDAC inhibitor is vorinostat, the PKC agonist is bryostatin-1 and the HDAC inhibitor is panobinostat, the PKC agonist is bryostatin-1 and the HDAC inhibitor is romidepsin, the PKC agonist is prostratin and the HDAC inhibitor is vorinostat, the PKC agonist is prostratin and the HDAC inhibitor is panobinostat, or the PKC agonist is prostratin and the HDAC inhibitor is romidepsin.

16. (canceled)

17. (canceled)

18. (canceled)

19. (canceled)

20. (canceled)

21. The method of claim 1, wherein the efficacy of the treatment results in an increase in the levels of intracellular HIV-1 mRNA compared to control.

22. The method of claim 21, wherein the increase in the levels of intracellular HIV-1 mRNA is measured as about 2-, about 4-, about 6-, about 8-, about 10-, about 12-, about 15-, about 20-, about 25-, about 30-, about 35-, about 40-, about 45-, about 50-, about 55-, about 60-, about 65-, about 70-, about 75-, about 80-, about 85-, about 90-, about 95-, or about 100-fold increase compared to control.

23. The method of claim 1, wherein the efficacy of the treatment results in an increase in the production and release of HIV-1 virions compared to control.

24. The method of claim 23, wherein the increase in the production and release of HIV-1 virions is measured as an average of about 1×10³, about 5×10³, about 1×10⁴, about 2×10⁴, about 4×10⁴, about 6×10⁴, about 8×10⁴, about 1×10⁵, about 2×10⁵, about 3×10⁵, about 4×10⁵, or about 5×10⁵ HIV-1 mRNA copies per milliliter of supernatant.

25. The method of claim 1, wherein the efficacy of the treatment results in (a) an increase in T cell activation, (b) an increase in the levels of plasma HIV-1 RNA, and/or (c) a reduction of the HIV-1 latent reservoir.

26. The method of claim 25, wherein the increase in T cell activation is measured as an increase in the production and release of at least one proinflammatory cytokine compared to control, wherein the proinflammatory cytokine is selected from the group consisting of TNF-α, IFN-γ, IL-2, IL-4, IL-6, IL-10, and IL-17.

27. (canceled)

28. (canceled)

29. The method of claim 25, wherein the increase of the levels of plasma HIV-1 RNA is in measure as about 1-log, about 2-logs, 3-logs, 4-logs, S-logs, 6-logs, 7-logs, 8-logs, or 9-logs increase compare to control.

30. (canceled)

31. The method of claim 25, wherein the HIV-1 latent reservoir is decreased 100-, 200-, 300-, 400-, 500-, 600-, 700-, 800-, 900-, 1000-, 1500-, or 2000-fold when compared to the resting CD4⁺ T cell population in any healthy or infected individual or total latently infected resting CD4⁺ T cell population.

32. The method of claim 31, wherein the resting CD4+ T cell population in any healthy or infected individual is about 10¹² cells.

33. The method of claim 31, wherein the total latently infected resting CD4+ T cell population is from about 10⁶ to about 10⁷ cells.

34. (canceled)

35. The method of claim 1, wherein the mammal is human, and said human is afflicted with HIV-1, chronically infected with HIV-1, or acutely infected with HIV-1.

36. (canceled)

37. (canceled)

38. The method of claim 35, wherein the combination therapy is administered to (a) a human on suppressive antiretroviral therapy, (b) an antiretroviral-treated human followed by antiretroviral treatment interruption, (c) a mammal more than one time over the course of treating or preventing, or (d) the mammal in need thereof immediately after said mammal is suppressed on antiretroviral therapy and at time after that for the remainder of the infected mammal's life.

39. (canceled)

40. (canceled)

41. (canceled)


SEQUENCE LISTING (3 sequences)

SEQ ID NO: 1 - DNA, 23 bp, Artificial Sequence
  synthetic primer
cagatgctgcatataagcagctg

SEQ ID NO: 2 - DNA, 31 bp, Artificial Sequence
  synthetic primer
ttttttttttttttttttttttttgaagcac

SEQ ID NO: 3 - DNA, 20 bp, Artificial Sequence
  synthetic primer
cctgtactgggtctctctgg